(19) United States
(12) Patent Application Publication
(10) Pub. No.: US 2019/0194604 A1
(43) Pub. Date: Jun. 27, 2019

(54) IN VITRO 3D CULTURE OF HUMAN BRAIN TISSUE

(71) Applicant: The Jackson Laboratory, Bar Harbor, ME (US)

(72) Inventor: Min Tang-Schomer, Marlborough, CT (US)

(73) Assignee: The Jackson Laboratory, Bar Harbor, ME (US)

(21) Appl. No.: 16/223,357

(22) Filed: Dec. 18, 2018

Related U.S. Application Data
(60) Provisional application No. 62/599,937, filed on Dec. 18, 2017.

(51) Int. Cl. C12N 5/00; C12Q 1/02; C12N 5/079

(52) U.S. Cl. CPC C12N 5/0062; C12N 5/0018; C12Q 1/025; C12N 5/0622; C12N 2533/32; C12N 2501/01; C12N 2501/11; C12N 2501/115; C12N 2501/165; C12N 2501/13; C12N 2533/90

(57) ABSTRACT

Provided herein, in some embodiments, are methods comprising culturing human brain tissue spheroids on a three-dimensional scaffold in culture media comprising vascular endothelial growth factor (VEGF), and producing a population of cells, wherein cells of the population are positive for nestin, glial fibrillary acidic protein (GFAP), and/or vimentin.

### RELATED APPLICATION

This application claims the benefit under 35 U.S.C. § 119(e) of U.S. provisional application No. 62/599,937, filed Dec. 18, 2017, which is incorporated by reference herein in its entirety.

### BACKGROUND

Human brain tissue is considered to be incapable of regeneration outside the body. Though animals are traditionally used to develop disease models for brain disorders, with the advent of advanced cell/tissue culture and stem/induced pluripotent (iPSC) cell technologies (Cairns et al., 2016; Lancaster et al., 2013; Lee et al., 2017), there is increasing interest in in vitro human brain tissue models. In vitro culture of primary neurons is widely adapted with embryonic, but not mature brain tissue.

### SUMMARY

Herein, a previously-developed bioengineered three-dimensional embryonic brain tissue model was applied to resected normal patient brain tissue in an attempt to regenerate human neurons in vitro. Single cells and small sized (diameter <100 μm) spheroids from dissociated brain tissue were seeded into three-dimensional silk fibroin-based scaffolds, with or without collagen or Matrigel™, and compared with two-dimensional cultures and scaffold-free suspension cultures. Changes of cell phenotypes (neuronal, astroglial, neural progenitor, and neuroepithelial) were quantified with flow cytometry and analyzed with a new method of statistical analysis specifically designed for percentage comparison. Compared with a complete lack of viable cells in conventional neuronal cell culture condition, supplements of vascular endothelial growth factor-containing pro-endothelial cell condition led to regenerative growth of neurons and astroglial cells from “normal” human brain tissue of epilepsy surgical patients. This process involved delayed expansion of nestin+ neural progenitor cells, emergence of TUJ1+ immature neurons, and Vimentin+ neuroepithelium-like cell sheet formation in prolonged cultures (14 weeks). Micro-tissue spheroids, but not single cells, supported the brain tissue growth, suggesting importance of preserving native cell-cell interactions. The presence of three-dimensional scaffold, but not hydrogel, allowed for Vimentin+ cell expansion, indicating a different growth mechanism than pluripotent cell-based brain organoid formation. The slow and delayed process implied an origin of quiescent neural precursors in the neocortex tissue. Further optimization of the three-dimensional tissue model with primary human brain cells could provide, for example, personalized brain disease models.

Thus, some aspects of the present disclosure provide methods comprising culturing human brain tissue spheroids on a three-dimensional scaffold in culture media comprising vascular endothelial growth factor (VEGF), and producing a population of cells, wherein cells of the population are positive for nestin, glial fibrillary acidic protein (GFAP), and/or vimentin.

Other aspects of the present disclosure provide compositions comprising human brain tissue spheroids, a three-dimensional scaffold, culture media comprising vascular endothelial growth factor (VEGF), and a population of cells, wherein cells of the population are positive for nestin, glial fibrillary acidic protein (GFAP), neuron-specific class III beta-tubulin (TUJI), and/or vimentin.

In some embodiments, the three-dimensional scaffold comprises silk fibroin. In some embodiments, the silk fibroin is prepared from Bombyx mori cocoons. In some embodiments, the three-dimensional scaffold is coated in polylysine. In some embodiments, the three-dimensional scaffold further comprises extracellular matrix (ECM) components.

In some embodiments, the three-dimensional scaffold has a length, width, and/or diameter of 2 mm to 10 mm, and/or a height of 2 mm to 10 mm. For example, the three-dimensional scaffold may have a length, width, and/or diameter of 2 mm to 8 mm, 2 mm to 6 mm, or 2 mm to 4 mm. In some embodiments, the three-dimensional scaffold has a length, width, and/or diameter of 2 mm, 3 mm, 4 mm, 5 mm, 6 mm, 7 mm, 8 mm, 9 mm, or 10 mm.

In some embodiments, the culture media further comprises Neurobasal™ Medium (ThermoFisher Scientific) or an equivalent medium (e.g., a medium have all the components of Neurobasal™ Medium). In some embodiments, the culture media further comprises amino acids, vitamins (e.g., choline chloride, D-calcium pantothenate, folic acid, niacinamide, pyridoxal hydrochloride, riboflavin, thiamine hydrochloride, vitamin b12, and/or i-inositol), inorganic salts (e.g., calcium chloride, ferric nitrate, magnesium chloride, potassium chloride, sodium bicarbonate, sodium chloride, sodium phosphate monobasic, and/or zinc sulfate), D-glucose, HEPES buffer, phenol red, sodium pyruvate, or any combination thereof.

In some embodiments, the culture media further comprises endothelial growth medium (EGM™). In some embodiments, the culture media further comprises EGM™-2 MV (Lonza), or an equivalent medium. In some embodiments the culture media further comprises a B-27 supplement, fibroblast growth factor (FGF), and/or epidermal growth factor (EGF). In some embodiments, the culture media further comprises Neurobasal™ Medium, EGM™-2 MV, B-27 supplement, FGF, and EGF.

In some embodiments, the concentration of VEGF is 1-5 ng/ml, for example, 1 ng/ml, 2 ng/ml, 3 ng/ml, 4 ng/ml, or 5 ng/ml.

In some embodiments, the concentration of FGF is 10-30 nm/ml, for example, 10 nm/ml, 20 nm/ml, or 30 nm/ml. In some embodiments, the concentration of EGF is 10-30 nm/mL, for example, 10 nm/ml, 20 nm/ml, or 30 nm/ml.

In some embodiments, the human brain tissue spheroids have a diameter of less than or equal to 100 μm. For example, the human brain tissue spheroids may a diameter of 20-100 μm, 30-100 μm, 40-100 μm, 50-100 μm, 60-100 μm, 70-100 μm, 80-100 μm, 20-80 μm, 30-80 μm, 40-80 μm, 50-80 μm, 20-60 μm, 30-60 μm, or 40-60 μm. In some embodiments, the human brain tissue spheroids have a diameter of 20 μm, 30 μm, 40 μm, 50 μm, 60 μm, 70 μm, 80 μm, 90 μm, or 100 μm.

In some embodiments, the human brain tissue spheroids are obtained (e.g., surgically) from a human neocortex or temporal lobe.

In some embodiments, the human brain tissue spheroids are cultured for at least 2 weeks, at least 4 weeks, or at least 6 weeks. In some embodiments, the human brain tissue spheroids are cultured for 2-6 week, 2-4 weeks, or 4-6 weeks. Longer culture times are contemplated herein.

In some embodiments, at least 30-50% (e.g., 30%, 40%, 50%, 60%, 70%, 80%, 90%, or 100%) of the cells of the population are positive (e.g., mRNA transcript positive and/or immunocytochemically positive) for nestin, at least 20-40% (e.g., 20%, 30%, 40%, 50%, 60%, 70%, 80%, 90%, or 100%) of the cells of the population are positive (e.g., mRNA transcript positive and/or immunocytochemically positive) for GFAP, and/or at least 10-25% (e.g., 10%, 15%, 20%, 25%, 35%, 40%, 50%, 60%, 70%, 80%, 90%, or 100%) of the cells of the population are positive (e.g., mRNA transcript positive and/or immunocytochemically positive) for vimentin.

In some embodiments, the methods further comprise replacing the culture media with neural differentiation media. In some embodiments, the neural differentiation media comprises Neurobasal™ Medium, B-27 supplement, cyclic adenosine monophosphate (cAMP), ascorbic acid, glial cell line-derived neurotrophic factor (GDNF), and/or brain-derived neurotrophic factor (BDNF) (e.g., any combination of two or more of the foregoing).

In some embodiments, the concentration of cAMP is 50-150 ng/ml (e.g., 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, or 150 ng/ml), the concentration of ascorbic acid is 0.5-2 μg/ml (e.g., 0.5 μg/ml, 1 μg/ml, 1.5 μg/ml, or 2 μg/ml), the concentration of GDNF is 5-20 ng/ml (e.g., 5 ng/ml, 10 ng/ml, 15 ng/ml, or 20 ng/ml), and/or the concentration of BDNF is 5-20 ng/ml(e.g., 5 ng/ml, 10 ng/ml, 15 ng/ml, or 20 ng/ml).

In some embodiments, the methods further comprise culturing the three-dimensional scaffold and the population of cells in the neural differentiation media. In some embodiments, the three-dimensional scaffold and the population of cells are cultured in the neural differentiation media for at least 4 weeks (e.g., 4-5, 4-6, 4-7, 4-8, 4-9, 4-10, 4, 5, 6, 7, 8, 9, or 10 weeks). In some embodiments, the methods further comprise producing cell clusters comprising cells that are positive for neuron-specific class III beta-tubulin (TUJ1) and GFAP.

In some embodiments, the method further comprises dissociating a human brain tissue sample to obtain the human brain tissue spheroids.

In some embodiments, the method further comprises culturing the human brain tissue spheroids in a suspension culture for at least 4 weeks (e.g., 4-5, 4-6, 4-7, 4-8, 4-9, 4-10, 4, 5, 6, 7, 8, 9, or 10 weeks), prior to culturing the human brain tissue spheroids on the three-dimensional scaffold.

Further aspects of the present disclosure provide methods of identifying/determining the effects of an agent on human brain tissue. In some embodiments, the methods comprise culturing human brain tissue spheroids on a three-dimensional scaffold in culture media comprising vascular endothelial growth factor (VEGF), and adding a test agent to the culture media, before, during, or after the culturing step. In some embodiments, the test agent is a small molecule drug or other molecule (e.g., nucleic acid (e.g., DNA or RNA) and/or protein/peptide) or compound (e.g., capable of crossing the blood-brain barrier. In some embodiments, the methods further comprise assaying/assessing the effects of the test agent on the human brain tissue. Characteristics that may be assessed include, for example, cell growth, proliferation, and/or differentiation, change in biomarker expression, and/or change in axonal growth rate and/or pattern. Other characteristics may be assessed.

Tang-Schomer MD et al., J Tissue Eng Regen Med. 2018; 12: 1247-1260 is incorporated herein by reference in its entirety.

### BRIEF DESCRIPTION OF THE DRAWINGS

FIGs. 1A-1B show the design of human patient brain tissue cultures. FIG. 1A depicts schematics of the study design, including the obtainment of human patient brain tissue, cell culture conditions, assessment, and expected outcomes. Human brain tissue is obtained from epileptic neurosurgical resection, mechanically dissociated into single cells and small spheroids (diameter <100 μm) and distributed for two-dimensional culture on polylysine (pll)-coated petri dishes, three-dimensional cultures on porous silk fibroin-based scaffolds, and suspension cultures of tissue spheroids. Different types of culture media (N, N +E and N+FBS) and ECM components (Matri., Col., Colg, ColgFN) are tested for assessing a range of cell/tissue growth parameters including morphology, phenotype, and viability, to determine an optimal combination of cell culture conditions for the three-dimensional growth of human patient brain tissue in vitro. Left, top: MRI T2 image of a 16-year-old boy undergoing right temporal lobe resection for intractable epilepsy. The resected brain region used for the study is marked. Left, bottom: The neocortex tissue delivered to the laboratory. Scale bar, 1 cm. Right, top: Phase contrast image of a two-dimensional culture at Day 7 in vitro (DIV 7). Right, center: Fluorescence image of a silk scaffold-based three-dimensional culture stained for live/dead at 24-hr postseeding. The scaffold material autofluoresces. Right, bottom: DAPI-stained tissue spheroids in a suspension culture at 24-hr postseeding. Right: scale bar, 50 μm. FIG. 1B shows a previous case of human patient's brain tissue culture stained with neuronal marker TUJ1 and glial marker GFAP. Scale bar, 100 μm.

FIGS. 2A-2E show human patient brain tissue in two-dimensional culture. FIG. 2A shows representative fluorescence images of two-dimensional cultures stained at DIV 3 weeks (top), 6 weeks (center), and 14 weeks (bottom) for neuronal marker TUJ1, astrocyte marker GFAP and neural progenitor cell marker, Nestin. The nuclei DAPI stain was used for all images. FIG. 2B shows flow cytometry quantification of cell percentages from trypsinized two-dimensional cultures and cell composition changes. Left: Percentage changes over time of Nestin+, GFAP+, and TUJ1+ cells of two-dimensional cultures in N+E media. Error bars show constructed simultaneous confidence intervals. Right: Histograms of positively stained cell population compared with unstained cells. Nestin, top; TUJ1, middle; GFAP, bottom. FIG. 2C shows a morphological assessment of TUJ1+ and GFAP+ cells in two-dimensional cultures in N+E media. Top: A representative fluorescence image of TUJ1/GFAP double positive process (white arrows). Scale bar, 100 μm. Bottom: Length measurement of TUJ1+ processes (left axis, bar graph) and the ratio of GFAP+ versus TUJ130 lengths of double positive segments (right axis line). At DIV 14 weeks, no double stained processes were found. Error bar, standard error of mean. N=9-20. **, p<0.01 compared with DIV 3 weeks. FIG. 2D shows an assessment of vimentin+ (Vim) cells in two-dimensional cultures in N+E media. Top: A representative fluorescence image of Vim-stained two-dimensional culture at DIV 6 weeks. Scale bar, 100 μm. Bottom: Flow cytometry quantification of Vim+ cell percentage changes in N+E (solid line) versus N media (dotted line). Error bars show constructed simultaneous confidence intervals. FIG. 2E shows an assessment of Ki67+ (mitosis marker) cells in two-dimensional cultures in N+E media. Top: A representative fluorescence image of Ki67-stained two-dimensional culture at DIV 5 weeks. Scale bar, 100 μm. Bottom: Flow cytometry quantification of Ki67+ cell percentage in N+E (solid line) versus N media (dotted line). Error bars show constructed simultaneous confidence intervals.

FIGS. 3A-3B show three-dimensional silk scaffold-based culture viability and DNA content. FIG. 3A depicts an alamarBlue® assay of 3D cultures over 8 weeks. Cultures in N+E (solid lines) show higher fluorescence intensity (Ex/Em 560/590) than in N (dotted line). Error bar, standard error of mean. N=2-4/group. **, p<0.01 compared with SF_N at corresponding time point. FIG. 3B shows DNA content measured from individual three-dimensional cultures with the Picogreen assay. Error bar, standard error of mean. N=3-4/group. *, p<0.05 compared with corresponding cultures in N media.

FIGS. 4A-4D show human patient brain cells in three-dimensional silk scaffold-only cultures. FIGS. 4A-4B show representative fluorescence images of neuronal (TUJ1+) and glial (GFAP+) processes in three-dimensional cultures in N+E media of DIV 1-3 weeks. FIG. 4A shows an example of fragmented TUJ1+ staining pattern (left top) and intact GFAP+ stained segment (left center) co-localization (left bottom, yellow). Right: A confocal image of congruous GFAP+ segments with beaded-looking TUJ1+ segments “wrapping” around (white arrows) the scaffold material that autofluoresces. Scale bar, 50 μm. FIG. 4B shows representative fluorescence images of the spiral wrapping pattern (white arrows) of GFAP+ staining in three-dimensional cultures (left, right-confocal). Scale bar, 50 μm. FIG. 4C shows representative fluorescence images of spheroid-looking cell clusters containing neural progenitor cells stained positive for NG2 (left, left center-confocal), Nestin (right center), and Vimentin (Vim) (right) among SmA+ cells. All cultures were in N+E media and stained at DIV 4-6 weeks. Scale bar, 100 μm. FIG. 4D shows representative histological images of paraffin sections of three-dimensional cultures at DIV 4-6 weeks. Haematoxylin and eosin staining showing cells along the scaffold surface (solid pink color) (left), and as clusters (center). Right: Vim-DAB staining. Scale bar, 100 μm.

FIGS. 5A-5C show human patient brain cells in three-dimensional cultures of silk scaffolds infused with ECM gels. FIG. 5A shows representative fluorescence images of smooth muscle actin (SmA+) and nestin (Nestin+) co-stained three-dimensional cultures of silk scaffolds infused with ECM gels. The cultures were stained at DIV 3 weeks. (top row) Positively stained cell bodies without processes in three-dimensional cultures of silk scaffolds infused with rat collagen type I gel added with EGM-2 MV SingleQuots without FBS (SF/Colg). (center row; bottom row, confocal) positively stained cells and processes in three-dimensional cultures of silk scaffold infused with Matrigel mixed at 1:1 with collagen type I gel (SF/Matri). Scale bar, 100 μm. FIG. 5B shows flow cytometry quantification of Nestin+ cell percentages in three-dimensional cultures. Error bars show constructed simultaneous confidence intervals. FIG. 5C shows a morphological assessment of three-dimensional cultures of ECM gel-infused silk scaffolds. The cultures were stained at DIV 4-6 weeks. (first column) Representative fluorescence images of smooth muscle actin (SmA+) and vimentin (Vim+) co-stained three-dimensional cultures of silk scaffolds infused with Colg (first column, top) or Matri (first column, bottom). (second and third columns, top and bottom,) Haematoxylin and eosin (H&E) staining of paraffin sections showing a sheet-like structure at the exterior surface of the scaffold (dotted line) and at the interface between the scaffold material and the ECM matrix (arrowhead), and cell invasion in Matrigel of SF/Matri cultures (third column, bottom, star). (fourth column) Vim-DAB staining showing Vim+ cells in the cell sheet-like structure at the scaffold surface (dotted line) and at the scaffold-matrix interface (arrowhead). Scale bar, 100 μm.

FIGS. 6A-6E show tissue spheroids in three-dimensional silk scaffolds to support regeneration-like three-dimensional growth of human patient brain tissue. FIG. 6A shows tissue spheroids in suspension cultures. Left and center: Representative fluorescence images of tissue spheroids embedded in Matrigel and cultured in N+E media for 4-5 weeks. Left: TUJ1+ and GFAP+ cell clusters without processes at DIV 4 weeks. (inset) Short GFAP+ processes at DIV 2 wk. Center: Nestin+ and Vim+ cell clusters and processes (white arrows, inset) at DIV 5 weeks. Scale bar, 100 μm. Right: Flow cytometry measured cell percentages of DIV 4 weeks spheroid cultures. Error bars show constructed simultaneous confidence intervals. FIG. 6B depicts a hypothetical illustration of different cell types of a neuronal lineage arising from a tissue spheroid that is housed within a three-dimensional scaffold. FIG. 6C depicts schematics of optimized human patient brain tissue culture. Dissociated human patient brain tissues are maintained as spheroids in suspension culture for a period of time (up to 6 weeks), seeded onto three-dimensional silk scaffolds and maintained in 96-well plates for long-term culture of another 6-8 weeks. FIG. 6D shows an assessment of three-dimensional silk scaffold-supported spheroid cultures. Tissue spheroids were in suspension culture for 6 weeks, seeded into silk scaffolds, and cultured in N+E for another 8 weeks.) Representative fluorescence images of cultures stained with TUJ1 , GFAP and DAPI, showing a cell sheet-like structure at the periphery of the scaffold (white arrows) are shown. Bright field is overlaid in the third and fourth images (top) to illuminate the underlying scaffold structure. The scaffold material autofluoresces. Scale bar, 100 μm. Bottom, right: Flow cytometry quantification of cell percentages of Nestin+, GFAP+, Vim+, and Ki67+ cells. N=3. Error bar, standard error of mean. FIG. 6E shows a morphological assessment of neuronal differentiation of three-dimensional silk scaffold-supported spheroid cultures. Tissue spheroids were in suspension culture for 6 weeks, seeded into silk scaffolds, cultured in N+E for two more weeks, and switched to neural differentiation media NDM+GFs for another 6 weeks. Representative fluorescence images of cultures stained with TUJ1 and GFAP are shown. Positively stained cells as clusters (third image, confocal) with extended cell processes (fourth image, confocal). Scale bar, 100 μm.

FIGS. 7A-7C show a Live/Dead stain of DIV 1-3 wk human patient brain tissue cultures. FIG. 7A shows two-dimensional cultures in different media, “N” (NeuralBasal/B27/bFGF/EGF) (first column), “N+FBS” (N supplemented with 10% fetal bovine serum) (second column), and “N+E” (N mix at 1:1 with EGM-2 MV media without FBS) (third column), and stained at DIV 6 (top row) and DIV 21 (bottom row). Note the fibroblast-looking cells in N+FBS. Scale bar, 100 μm. FIG. 7B shows a suspension culture of tissue spheroids as spheroid-only, “Spheroid” (left), or embedded in rat collagen type I, “Spheroid/Col” (center), or in Matrigel mixed at 1:1 with rat collagen type I, “Spheroid/Matri” (right). Cultures were stained at DIV 6. The suspension culture contained live spheroids (marked with *) and dead spheroids (red, arrowhead). Note cell extensions in the matrix in Spheroid/Matri cultures (left, white arrow), but not in other spheroid cultures. Scale bar, 100 μm. FIG. 7C shows three-dimensional cultures of scaffold-only “SF” (first column), scaffold infused with collagen mixed in EGM-MV supplements “SF/Colg” (second column), and scaffold infused with Matrigel “SF/Matri” (third column), inN (top row) and N+E (bottom row) media. Cultures were stained at DIV 21. Scaffold material autofluoresces. Note that live cells were contained in clusters (marked with dotted lines). Scale bar, 100 μm.

FIG. 8 shows fluorescence images of two-dimensional cultures of human patient brain tissue culture. GFAP+ cells (top left and center). NG2+ cells (top right). Smooth muscle actin (SmA)+ cells (bottom left). SmA and Nestion double-stain (bottom center). NG2 and PDGF receptor beta double-stain (bottom right).

FIG. 9 shows linear correlation of DNA content and cell numbers. DNA content was measured with the Picogreen assay. Cells were plated in two-dimensional cultures with controlled cell numbers for 24 hours, and extracted for DNA measurements. Human cerebral microvascular cell line hMEC and glioblastoma cell line U87 were used.

FIGS. 10A-10B show flow cytometry quantification of cell percentages of three-dimensional silk scaffold-based cultures. FIG. 10A shows TUJ1+ cell percentage. FIG. 10B shows GFAP+ cell percentage.

FIG. 11 shows the first patient's brain cells in three-dimensional silk scaffold-only cultures. The cultures were stained with GFAP at DIV 1 wk. Representative fluorescence images of the spiral wrapping pattern of GFAP+ staining around the silk scaffold structure (white arrows). The silk material autofluoresces. Scale bar, 100 μm.

### DETAILED DESCRIPTION

The advancement of three-dimensional tissue engineering provides new avenues for developing human brain tissue models. A three-dimensional architecture is important for cells to interact with each other and with the extracellular matrix (ECM). As demonstrated in recently developed three-dimensional brain cortical tissue models with rat embryonic neurons, neuronal clusters anchored on a porous scaffold surface can extend long-distance (in millimeters) axonal connections through permissive rat collagen type I gel matrix (Chwalek, Sood, et al., 2015; Chwalek, Tang-Schomer, Omenetto, & Kaplan, 2015; Tang-Schomer et al., 2014, each of which is incorporated herein by reference). This three-dimensional in vitro culture system showed sustained long-term tissue viability and exhibited in vivo-like biochemical and electrophysiological responses to mechanical perturbation simulating traumatic brain injury. The three-dimensional scaffold is made of silk fibroin solution prepared from Bombyx mori (commonly known as silkworm) cocoons (Altman et al., 2003). The versatility of the silk material processing allowed us to generate a scaffold base that has mechanical properties similar to the native brain tissue (Tang-Schomer et al., 2014). As an inert biomaterial, the silk fibroin-based scaffold base does not react with neurons, but addition of a polylysine coating permits neuronal attachment. The porous structure allows infusion of exogenous ECM components to produce a genuine three-dimensional microenvironment; permitting separate examinations of bio-active components, such as ECM protein types (Sood et al., 2016), from the properties of a three-dimensional structure, such as stiffness and shape (Rockwood et al., 2011; Tang-Schomer et al., 2014).

Three-dimensional in vitro culture systems combined with a patient's cells can offer an opportunity to recapitulate tissue-level physiology to compare with the patient's clinical findings. Human patient brain tissue can be obtained from neurosurgical procedures for intractable epilepsy, with estimated 4,000 cases per year performed in the United States (Gumnit, Labiner, Fountain, & Herman, 2012). These tissues are considered “normal” due to their non-tumorigenic genetic background and normal magnetic resonance imaging findings. At the Connecticut Children's Medical Center (CCMC), there were two subjects in 2015 that required neurosurgical removal of the temporal lobe tissue. In this study, the objective was to extend the previously developed three-dimensional brain tissue model to normal human patient brain tissue and establish optimal cell culture conditions for growing human neurons. Dissociated cells from surgically resected brain tissue were seeded onto the three-dimensional silk scaffold-based model and compared outcomes with conventional two-dimensional cultures and scaffold-free suspension cultures of tissue spheroids. These different forms of culture systems were used to examine roles of medium types, ECM components, substrates, and cell initial states on tissue growth with regard to cell phenotype changes and morphology. The results show that with optimized microenvironmental factors, it is possible to grow neurons derived from human patient brain tissue in three-dimensional in vitro systems.

The present disclosure provides a method comprising culturing human brain tissue spheroids on a three-dimensional scaffold in culture media comprising vascular endothelial growth factor (VEGF). Culturing refers to maintaining and/or propagating cells or spheroids in vitro. In some embodiments, cells are isolated from a human subject (e.g., brain tissue). In some embodiments, the method produces a population of cells, wherein cells of the population are positive for nestin, glial fibrillary acid protein (GFAP), and/or vimentin.

In some embodiments, the present disclosure provides a composition comprising human brain tissue spheroids, a three-dimensional scaffold, culture media comprising VEGF, and a population of cells, wherein cells of the population are positive for nestin, GFAP, and/or vimentin.

### Spheroid Cultures

The present disclosure provides a method for dissociating and culturing human brain tissue spheroids on a three-dimensional scaffold. Dissociation is the isolation of single cells and spheroids from a tissue sample. Non-limiting examples of methods for dissociating brain tissue include but are not limited to heating, gentle agitation, enzymatic digest (e.g., collagenase, trypsin, papain, hyaluronidase, or deoxyribonuclease), and straining. Spheroids are three-dimensional multi-cellular structures containing cells and extracellular matrix that more closely mimic in vivo conditions than traditional two-dimensional cell cultures. Spheroids may be isolated from human tissue samples or constructed in vitro. Spheroids and/or cells may be isolated from any solid human tissue sample (e.g., muscle, brain, bone, liver, pancreas, stomach, colon, small intestine, kidney, etc.). In some embodiments, spheroids and/or cells are isolated from brain tissue. Non-limiting examples of brain tissues are neocortex, temporal lobe, cerebrum, cerebellum, brain stem, cortex, thalamus, hypothalamus, parietal lobe, frontal lobe, myelencephalon, mesencephalon, epithalamus, subthalamus, and pituitary gland. In some embodiments, the human brain tissue spheroids are obtained from a human neocortex or temporal lobe.

In some embodiments, spheroids are cultured prior to being seeded onto three-dimensional scaffolds. Culturing refers to maintaining cells and/or spheroids in vitro in media under defined conditions. Culturing may be stationary (e.g., in dishes) or suspension. Suspension is a liquid culture comprising cells and/or spheroids that are agitated. Non-limiting examples of suspension culturing include shaking, rotating, and circulating.

In some embodiments, the spheroids are cultured in suspension culture for at least 4 weeks prior to culturing the human brain tissue spheroids on the three-dimensional scaffold. In some embodiments, the spheroids are cultured for 2 weeks-8 weeks prior to culturing the human brain tissue spheroids on the three-dimensional scaffold. In some embodiments, the spheroids are cultured for 1 week-12 weeks prior to culturing the human brain tissue spheroids on the three-dimensional scaffold. In some embodiments, the spheroids are cultured for 1 week, 2 weeks, 3 weeks, 4 weeks, 5 weeks, 6 weeks, 7 weeks, 8 weeks, 9 weeks, 10 weeks, 11 weeks, or 12 weeks prior to culturing the human brain tissue spheroids on the three-dimensional scaffold.

In some embodiments, the human brain tissue spheroids are cultured for at least 2 weeks, at least 4 weeks, or at least 6 weeks on a three-dimensional scaffold. In some embodiments, the human brain tissue spheroids are cultured for at least 2 weeks on a three-dimensional scaffold. In some embodiments, the human brain tissue spheroids are cultured for 1 weeks-12 weeks on a three-dimensional scaffold. In some embodiments, the human brain tissue spheroids are cultured for 1 week, 2 weeks, 3 weeks, 4 weeks, 5 weeks, 6 weeks, 7 weeks, 8 weeks, 9 weeks, 10 weeks, 11 weeks, or 12 weeks on a three-dimensional scaffold.

In some embodiments, the human brain tissue spheroids have a diameter of less than or equal to 100 μM. In some embodiments, the human brain tissue spheroids have a diameter of 50 μM-500 μM. In some embodiments, the human brain tissue spheroids have a diameter of 100 μM-250 μM. In some embodiments, the human brain tissue spheroids have a diameter of 50 μM, 75 μM, 100 μM, 125 μM, 150 μM, 175 μM, 200 μM, 225 μM, 250 μM, 275 μM, 300 μM, 325 μM, 350 μM, 375 μM, 400 μM, 425 μM, 450 μM, 475 μM, or 500 μM.

### Culture Medium

Cells and/or spheroids are cultured in vitro in media. Media refers to a chemically defined liquid in which cells and/or spheroids are maintained. Non-limiting examples of media include Neurobasal™, endothelial growth medium (EGM™), EGM™-2MV, Neuron Basal Medium™, BrainPhys™, Dulbecco's Modified Eagle Medium (DMEM™), Basal Medium Eagle (BME™), and Roswell Park Institute Medium (RPMI™). In some embodiments, the cells and/or spheroids are maintained in Neurobasal™ or an equivalent medium.

Compounds, proteins, drugs, and/or other agents may be added to media to regulate the cells and/or spheroids. Non-limiting examples of compounds, proteins, drugs, and/or other agents that may be added include vascular endothelial growth factor (VEGF), B-27 supplement, fibroblast growth factor (FGF), epidermal growth factor (EGF), cyclic adenosine monophosphate (cAMP), ascorbic acid, glial cell line-derived neurotrophic factor (GDNF), brain derived neurotrophic factor (BDNF), ampicillin, penicillin, streptomycin, gentamicin, glucose, insulin, N2™, N21™, bovine serum albumin (BSA), and GS21™.

In some embodiments, the cell and/or spheroid culture medium comprises Neurobasal™ or an equivalent medium. In some embodiments, the medium further comprises VEGF. In some embodiments, the medium further comprises EGM™. In some embodiments, the culture media further comprises EGM™-2MV or an equivalent medium. In some embodiments, the medium further comprises B-27 supplement, FGF, and/or EGF. In some embodiments, the culture medium comprises VEGF, Neurobasal™ Medium, EGM™-2MV, B-27 supplement, FGF, and EGF.

In some embodiments, the medium comprises 1-5 ng/mL VEGF. In some embodiments, the medium comprises 2 ng/mL VEGF. In some embodiments, the medium comprises 1-20 ng/mL VEGF. In some embodiments, the medium comprises 2-16 ng/mL VEGF. In some embodiments, the medium comprises 1, 2, 3, 4, 5, 6, 7, 8, 9, 10, 11, 12, 13, 14, 15, 16, 17, 18, 19, or 20 ng/mL VEGF. In some embodiments, the medium comprises less than 20 ng/mL VEGF. In some embodiments, the medium comprises at least 1 ng/mL VEGF.

In some embodiments, the medium comprises 10-30 ng/mL FGF. In some embodiments, the medium comprises 20 ng/mL FGF. In some embodiments, the medium comprises 5-50 ng/mL FGF. In some embodiments, the medium comprises 10-40 ng/mL FGF. In some embodiments, the medium comprises 5, 10, 15, 20, 25, 30, 35, 40, 45, or 50 ng/mL FGF. In some embodiments, the medium comprises 10-30 ng/mL EGF. In some embodiments, the medium comprises 20 ng/mL EGF. In some embodiments, the medium comprises 5-50 ng/mL EGF. In some embodiments, the medium comprises 10-40 ng/mL EGF. In some embodiments, the medium comprises 5, 10, 15, 20, 25, 30, 35, 40, 45, or 50 ng/mL EGF.

In some embodiments, the culture medium comprising VEGF, Neurobasal™ Medium, EGM™-2MV, B-27 supplement, FGF, and EGF is replaced with a neural differentiation medium. Neural differentiation medium comprises compounds, proteins drugs, and/or other agents that promote the differentiation of stem cells into differentiated neurons and/or neuron-like cells. In some embodiments, the neural differentiation medium comprises Neurobasal™ Medium, B-27 supplement, cAMP, ascorbic acid, GDNF, and/or BDNF.

In some embodiments, the neural differentiation medium comprises 50-150 ng/mL cAMP. In some embodiments, the neural differentiation medium comprises 25-200 ng/mL cAMP. In some embodiments, the neural differentiation medium comprises 10-100 ng/mL cAMP. In some embodiments, the neural differentiation medium comprises 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 ng/mL cAMP. In some embodiments, the neural differentiation medium comprises at least 50 ng/mL cAMP. In some embodiments, the neural differentiation medium comprises less than 200 ng/mL cAMP.

In some embodiments, the neural differentiation medium comprises 0.5-2.0 μg/mL ascorbic acid. In some embodiments, the neural differentiation medium comprises 1.0-2.0 μg/mL ascorbic acid. In some embodiments, the neural differentiation medium comprises 0.25-4.0 μg/mL ascorbic acid. In some embodiments, the neural differentiation medium comprises 0.25, 0.50, 0.75, 1.00, 1.25, 1.50, 1.75, 2.00, 2.25, 2.50, 2.75, 3.00, 3.25, 3.50, 3.75, or 4.00 μg/mL ascorbic acid. In some embodiments, the neural differentiation medium comprises at least 0.5 μg/mL ascorbic acid. In some embodiments, the neural differentiation medium comprises less than 2.0 μg/mL ascorbic acid.

In some embodiments, the neural differentiation medium comprises 5-15 ng/mL GDNF. In some embodiments, the neural differentiation medium comprises 1-30 ng/mL GDNF. In some embodiments, the neural differentiation medium comprises 1-15 ng/mL GDNF. In some embodiments, the neural differentiation medium comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL GDNF. In some embodiments, the neural differentiation medium comprises at least 5 ng/mL GDNF. In some embodiments, the neural differentiation medium comprises less than 15 ng/mL GDNF.

In some embodiments, the neural differentiation medium comprises 5-15 ng/mL BDNF. In some embodiments, the neural differentiation medium comprises 1-30 ng/mL BDNF. In some embodiments, the neural differentiation medium comprises 1-15 ng/mL BDNF. In some embodiments, the neural differentiation medium comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL BDNF. In some embodiments, the neural differentiation medium comprises at least 5 ng/mL BDNF. In some embodiments, the neural differentiation medium comprises less than 15 ng/mL BDNF.

### Three-Dimensional Scaffolds

A three-dimensional scaffold is a structure that is designed to mimic in vivo conditions of a tissue. Three-dimensional scaffolds are typically composed of porous, biocompatible, and biodegradable materials that serve to provide suitable mechanical support, physical, and biochemical stimuli for optimal cell growth and function. Non-limiting examples of three-dimensional scaffolds include hydrogels, tubes, sponges, composites, fibers, microspheres, and thin films.

The porosity and pore size of three-dimensional scaffolds has direct implications on the functionality of the scaffold. Open porous surfaces and interconnected networks of scaffold components are important for cell nutrition, proliferation, tissue vascularization, and formation of new tissues. Materials with high porosity also enable the effective uptake and release of soluble factors, such as proteins and nucleic acids, into and out of cells.

In some embodiments, the three-dimensional scaffold has a length, width, and/or diameter of 2 mm to 10 mm, and/or a height of 2 mm to 10 mm. In some embodiments, the three-dimensional scaffold has a length, width, and/or diameter of 2 mm to 20 mm. In some embodiments, the three-dimensional scaffold has a length, width, and/or diameter of 2 mm to 25 mm. In some embodiments, the three-dimensional scaffold has a length, width, and/or diameter of 1 mm to 50 mm. In some embodiments, the three-dimensional scaffold has a length, width, and/or diameter of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm. In some embodiments, the three-dimensional scaffold has a length, width, and/or diameter of at least 2 mm. In some embodiments, the three-dimensional scaffold has a length, width, and/or diameter of less than or equal to 50 mm.

In some embodiments, the three-dimensional scaffold has height of 2 mm to 25 mm. In some embodiments, the three-dimensional scaffold has a height of 1 mm to 50 mm. In some embodiments, the three-dimensional scaffold has a height of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm. In some embodiments, the three-dimensional scaffold has a height of at least 2 mm. In some embodiments, the three-dimensional scaffold has a height of less than or equal to 50 mm.

A three-dimensional scaffold can be composed of naturally-occurring materials, man-made materials, or a mixture of naturally-occurring and synthetic materials. Examples of scaffold materials include, without limitation, minerals (e.g., hydroxyapatite), proteins (e.g., elastin, alginate, albumin, fibroin, and collagen), metals (e.g., titanium, gold), and composites (e.g., poly(lactic-co-glycolic acid)/poly(c-caprolactone) PLGA/PCL, halloysite nanotubes).

In some embodiments, a three-dimensional scaffold comprises a naturally-occurring material Bombyx mori silk fibroin protein. Bombyx mori is a silkworm whose cocoons contain the silk fibroin protein. Silk fibroin is used in numerous biomaterial applications because it is biocompatible with in vivo models, it has controllable degradation rates that range from hours to years, and it can be chemically modified to altering surface properties of the three-dimensional scaffold or to immobilize growth factors. See, e.g., Tang-Schomer et al. PNAS 2014; 111(38): 13811-13816, incorporated herein by reference.

In some embodiments, a three-dimensional scaffold is coated with at least one compound that promotes adherence of cells, spheroids, and/or tissues. Non-limiting examples of compounds used to coat three-dimensional scaffolds include polylysine, polyornithine, collagen, polystyrene, gelatin, and fibronectin. In some embodiments, a three-dimensional scaffold is coated with polylysine.

### Extracellular Matrix

In some embodiments, the three-dimensional scaffold further comprises extracellular matrix components. Extracellular matrix (ECM) is the collection of molecules secreted by cells into the extracellular space. The ECM is composed of functional proteins (e.g., collagen and elastin), proteoglycans (e.g., heparan sulfate, chondroitin sulfate, and keratan sulfate), non-proteoglycan polysaccharides (e.g., hyaluronic acid) and a variety of growth factors, cytokines, ions, and water that provide structural support to cells. The composition of ECM varies between tissues, but the ECM regulates cell proliferation, differentiation, and migration.

One function of a three-dimensional scaffold is to mimic the actual ECM of the tissue. Different tissues have their own unique ECM compositions and structures, but in general the ECM is a porous meshwork of proteins and glycosaminoglycans that can be remodeled by cross-linking of collagen and elastin, wherein an increase in cross-linking decreases the porosity of the ECM. Remodeling of the ECM is important to processes such as cellular development, wound repair, and morphogenesis.

The ECM of the adult human brain is highly organized and unique in composition compared to other tissues. The brain ECM contains low levels of fibrous proteins and mainly includes aggregating proteoglycans, allowing a less rigid structure than other tissues. The composition, quantity, and structure of the ECM in the brain changes dramatically during the development of a mammal. The fetal brain ECM includes a higher level of fibrous proteins and a lower level of aggregating proteoglycans compared with the adult brain, which supports the growth of axons and the interconnection of neurons.

In some embodiments, the ECM comprises hyaluronic acid. Hyaluronic acid is a polysaccharide consisting of alternating residues of D-glucouronic acid and N-acetylglucosamine. Hyaluronic acid confers the ability to resist compression of the ECM by being able to absorb significant amounts of water. Hyaluronic acid acts as a regulator of cell behavior including embryonic development, healing, inflammation, and tumor development.

In some embodiments, the ECM comprises soluble factors present in brain tissue. Soluble factors are molecules in tissue that regulate cell growth, cell proliferation, cell division, inflammation, angiogenesis and tumorigenesis. Non-limiting examples of soluble factors in the brain include vascular endothelial growth factor (VEGF), interleukin (IL)-10, prostaglandin-2 (PGE-2), interleukin (IL)-6, interleukin (IL)-1α, fibroblast growth factor (FGF), hepatocyte growth factor (HGF), heparanase, matrix metalloproteases (MMPs). In some embodiments, the ECM comprises structural proteins present in brain tissue. Structural proteins are molecules in brain tissue which provide structure, cell-cell contacts, and cell-cell communication. Non-limiting examples of structural proteins include laminin, nidogen, collagen, and elastin.

### Neural Markers

The present disclosure provides a method comprising culturing human brain tissue spheroids and producing a population of cells, wherein the cells are positive for expression of neural markers. Neural markers are molecules on the surface of neurons, glial cells, and neuron-like cells (e.g., stem cells and neuroepithelial cells). Glial cells are non-neuronal cells in the CNS that maintain homeostasis, form myelin, and provide support and protection for the neurons. Non-limiting examples of glial cells include oligodendrocytes, astrocytes, ependymal cells, Schwann cells, microglia, and satellite cells. Neural markers may not only vary by the type of cell, but also by the stage of differentiation in neurons, neuron-like, and/or glial cells.

In some embodiments, neural markers are used to identify neurons, neuron-like cells, and/or glial cells, as well as the stage of neuron differentiation. In some embodiments, neural markers are bound by proteins (e.g., antibodies, preferably fluorescent antibodies) to classify neurons and neuron-like cells. Non-limiting examples of neural markers include nestin, SOX2, notch1, HES1, HES3, vimentin, PAX6, glial fibrillary acidic protein, TBR2, beta III tubulin, NG2, Nurr1, LMX1B, FOXA2, Pet1, NMDAR2B, vGluT1, vGluT2, MASH1/Ascl1, OSP, MOG, and SOX10.

In some embodiments, the population of cells express the neural marker protein nestin. Nestin (NP_006608.1) is a filament protein expressed from the NES gene (Gene ID: 10763) in dividing cells during early development of the central nervous system (CNS), peripheral nervous system, and other tissues. Nestin is a neural stem cell/progenitor cell marker and upon differentiation, nestin expression is down-regulated and replaced by other filament proteins.

In some embodiments, the population of cells express the neural marker protein glial fibrillary acidic protein (GFAP). GFAP (NP_001124491.1, NP_001229305.1, NP_001350775.1, or NP_002046.1) is a filament protein expressed from the GFAP gene (Gene ID: 2670) in numerous glial cells of the CNS, including astrocytes and ependymal cells. GFAP is involved in numerous CNS processes, including cell-cell contact, cell-cell communication and the integrity of the blood-brain barrier.

In some embodiments, the population of cells express the neural marker protein vimentin. Vimentin (NP_003371.2) is a filament protein expressed from the VIM gene (Gene ID: 7431) in cells throughout the human body, and it is critical in maintaining cell shape, the integrity of the cytoplasm, and stabilizing cytoskeletal interactions. In the CNS, vimentin is involved in the formation of new axons and dendrites which extend from the cell body of the neuron.

In some embodiments, the population of cells express the neural marker protein beta III tubulin (TUJ1). TUJ1 (NP_001184119.1 and NP_006077.2) is a microtubule protein expressed from the TUBB3 (Gene ID: 10381) in neurons. In the CNS, TUJ1 is involved in neurogenesis, axon guidance, and axon maintenance.

In some embodiments, the present disclosure provides a method comprising producing cell clusters comprising cells that are positive for neuron-specific TUJ1 and GFAP. In some embodiments, the cells of the cell clusters comprise extended processes (e.g., axons, or immature axons).

In some embodiments, at least 30-50% of the cells of the population are positive for nestin. In some embodiments, at least 20-60% of the cells of the population are positive for nestin. In some embodiments, at least 30-60% of the cells of the population are positive for nestin. In some embodiments, at least 20%, 25%, 30%, 35%, 40%, 45%, 50%, 55%, or 60% of cells of the population are positive for nestin.

In some embodiments, at least 20-40% of the cells of the population are positive for GFAP. In some embodiments, at least 10-60% of the cells of the population are positive for GFAP. In some embodiments, at least 20-50% of the cells of the population are positive for GFAP. In some embodiments, at least 10%, 15%, 20%, 25%, 30%, 35%, 40%, 45%, 50%, 55%, or 60% of cells of the population are positive for GFAP.

In some embodiments, at least 10-25% of the cells of the population are positive for vimentin. In some embodiments, at least 5-40% of the cells of the population are positive for vimentin. In some embodiments, at least 10-35% of the cells of the population are positive for vimentin. In some embodiments, at least 5%, 10%, 15%, 20%, 25%, 30%, 35%, or 40% of cells of the population are positive for vimentin.

In some embodiments, the present disclosure provides a method comprising culturing human brain tissue spheroids on a three-dimensional scaffold in culture media comprising VEGF and producing a population of cells, wherein at least 30-50% of the cells are positive for nestin, at least 20-40% of the cells of the population are positive for GFAP, and/or at least 10-25% of the cells of the population are positive for vimentin.

### EXAMPLES

Post-mitotic brain tissues cannot survive in conventional two-dimensional cultures. Here, three-dimensional tissue engineering methods was applied to surgically resected brain biopsies in an attempt to regenerate human patient brain tissue in vitro. A new statistical method was developed to compare group differences of cell percentages for the assessment of cell phenotype responses to different culture conditions. Together with morphological analysis, a combination of small tissue spheroids (diameter <100 μm), three-dimensional porous silk-based scaffolds, and VEGF-containing pro-endothelial cell growth media mixed with Neuralbasal/B27/EGF/FGF generates human neurons in three-dimensional in vitro cultures. This process involved delayed expansion of neuroepithelial-like cells and neural progenitor cells, emergence of immature neurons in prolonged tissue cultures (up to 14 weeks), and neuroepithelial-like cell sheet formation, with features remnant of neurogenesis in development.

### Example 1

### Study Design for Development of Cell Culture Conditions of Human Patient Brain Tissue

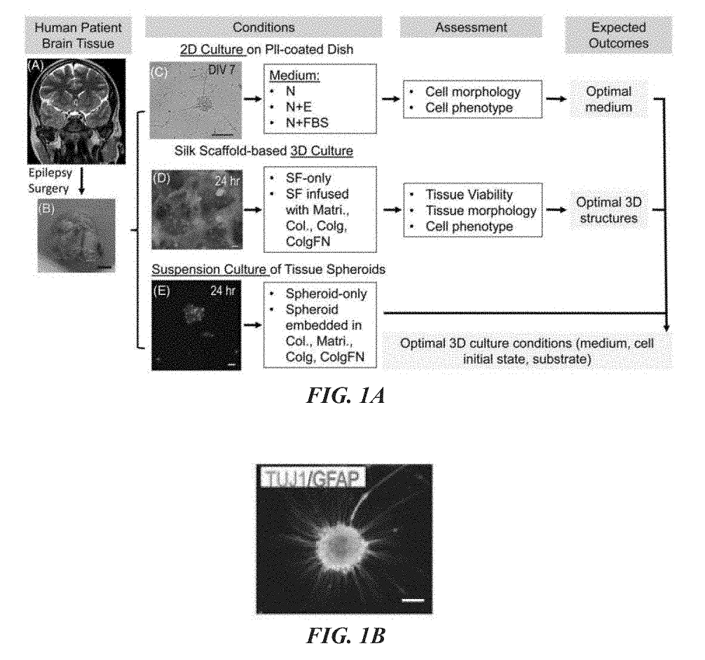

Human brain tissue was obtained from epileptic neurosurgical resection with the patient's consent, under CCMC IRB 15-033. Tissue was mechanically dissociated into single cells and small spheroids (diameter <100 μm) and distributed for in vitro cell/tissue culture. The first brain tissue sample was from a 13-year-old girl's temporal lobe resection, and was cultured in NeuralBasal/B27 media with or without fetal bovine serum (FBS). Almost all cells (>100 million cells tested) were dead within a week in culture; however, group of viable cultures were maintained in NeuralBasal/B27 media supplemented with vascular endothelial growth factor (VEGF)-containing endothelial growth media EGM-2 MV. These cultures presented neural cell clusters with positive neuronal (TUJ1+) and astroglial (GFAP+) markers in the cell bodies as well as processes (FIG. 1B).

The study design for the development of cell culture conditions is illustrated in FIG. 1A, including the obtainment of human patient brain tissue, cell/tissue culture conditions, assessment, and expected outcomes. Different types of culture media, extracellular matrix (ECM) components, and the cell initial state (i.e., as single cells or spheroids) were tested with a range of cell/tissue growth parameters including morphology, phenotype, and viability, to develop a combination of cell culture conditions for generating neurons in three-dimensional cultures from a patient's brain tissue.

The second brain tissue sample was from the resected brain tissue of a 16-year-old boy undergoing epilepsy surgery. The tissue was considered “normal” based on non-oncological features and normal magnetic resonance T2 imaging (FIG. 1A (left, to image), resected area marked). The neocortex tissue was delivered to the laboratory within 4-hr post-resection (FIG. 1B (left, bottom image)).

Dissociated cells were divided into three tissue culture groups: “two-dimensional culture” on poly-L-lysine (pll)-coated polystyrene plates, “three-dimensional culture” on pll-coated porous silk scaffolds as described in the previously developed brain tissue model with rat embryonic cortical cells (Tang-Schomer et al., 2014), and “suspension culture” of tissue spheroids. Three media conditions were tested: NeuralBasal/B27 supplemented with fibroblast growth factor (FGF, 20 ng/ml) and epidermal growth factor (EGF, 20 ng/ml) as used for neural stem cell expansion (“N”), NeuralBasal/B27/EGF/FGF supplemented with 10% FBS (“N+FBS”), and “N” media mixed at 1:1 ratio with VEGF-containing EGM-2 MV without serum (“N+E”).

To examine the effects of ECM components, the silk scaffold-based (“SF”) three-dimensional cultures and suspension cultures of tissue spheroids were divided into groups and exposed to different types of ECM gels after 3 days in vitro (DIV 3). ECM gels included rat collagen type I (“Col”), Matrigel mixed at 1:1 ratio with rat collagen type I (“Matri”), rat collagen type I supplemented with EGM-2 MV SingleQuots without FBS (including hydrocortisone, hFGF-B, VEGF, R3-IGF-1, ascorbic acid, and hEGF; “Colg”), and Colg supplemented with fibronectin (10μg/m1),(“ColgFN”).

Because there was a finite cell quantity of the initial human brain tissue sample, and due to the large numbers of variables to be tested and the destructive nature of the endpoint assays (i.e., immunostaining and flow cytometry), the sample size per assessment was limited. For this reason, an adaptive approach was taken. The study was divided into three phases, 0-3,3-6, and >6 weeks. More assessments were performed for the earlier time-points' cultures, and the test parameter range was narrowed for later time point assessments. The optimal conditions determined from previous phases were applied to the remaining live cultures in the last phase. The study was initiated with >500 cultures.

### Example 2

### Construction of Simultaneous Confidence Intervals for Comparing Cell Percentages of Multiple Groups

Flow cytometry of cell type-specific marker was used to track cell phenotype changes overtime. In order to compare group differences on a binary response, that is, positive or negative staining of a cell, confidence intervals of each response rate are calculated. A statistical method that is used for the analysis of patient population differences in clinical trials was adapted (Agresti, Bini, Bertaccini, & Ryu, 2008). Given the significance level α=0.05 and observed percentages, \hat{p}₁, \hat{p}₂, . . . \hat{p}_{N}, each of which is observed and calculated from n (n=1,000 for this study) experiments (i.e., flow cytometry events),Wilson's method was applied (Agresti & Coull, 1998; Brown, Cai, & DasGupta, 2001) to construct simultaneous confidence intervals.

The cut-off value chosen was z=z_{1-α/(2N)}, which is the

$$( 1 - \frac{a}{2 N} )$$

th quantile of a standard normal distribution; N is the sample size of all data points for comparison. If the cut-off value chosen was z=z_{1-α/2}, then the solutions would give point-wise confidence intervals, which are narrower than the simultaneous intervals desired. Note that comparisons were made for multiple percentages; a simultaneous version is needed as it adjusts for multiplicity.

Based on the solutions Îᵢ, ûᵢ the equation

$${( {\hat{p}}_{i} - x )}^{2} = \frac{{z}^{2} x ( 1 - x )}{n} . \qquad ( 1 )$$

The simultaneous confidence intervals were constructed for pᵢ as [symbol] . Note that Equation 1 has the close form solution

$$x = \frac{2 {\hat{p}}_{i} + \frac{{z}^{2}}{n} \pm \sqrt{{( 2 {\hat{p}}_{i} + \frac{{z}^{2}}{n} )}^{2} - 4 ( 1 + \frac{{Z}^{2}}{n} ) {\hat{p}}_{i}^{2}}}{2 ( 1 + \frac{{Z}^{2}}{n} )} . \qquad ( 2 )$$

All cell percentage data points were presented with corresponding confidence intervals as the error bars in the charts. Two measurements are considered significantly different if their error bars do not overlap.

### Example 3

### Initial Survival of Human Patient Brain Tissue In Vitro

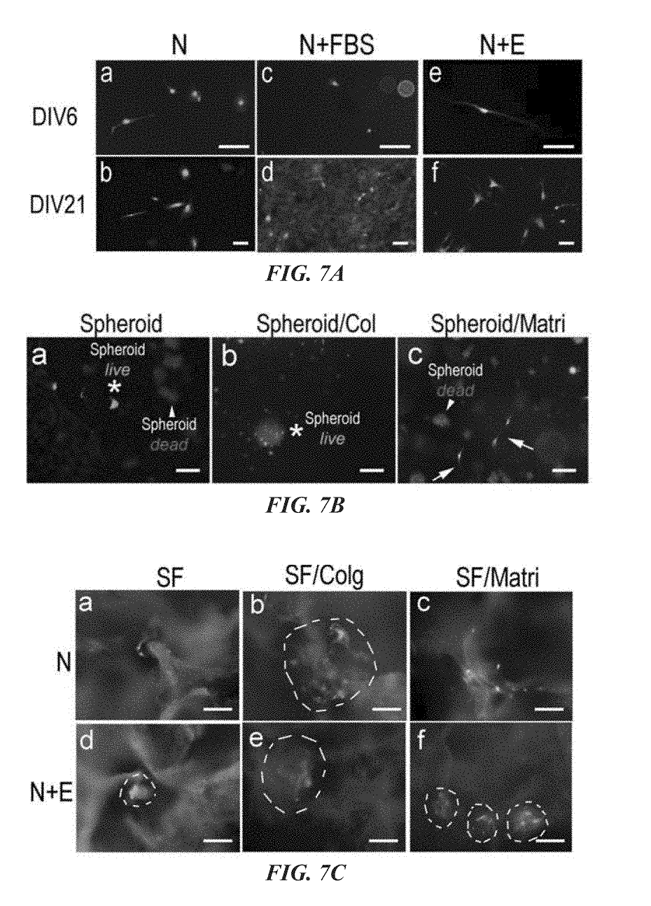

Unlike embryonic cortical neurons that exhibited axonal outgrowth within hours after plating, a majority of human patient brain cells remained devoid of processes in two-dimensional cultures in the first week (>30 six-well plates inspected daily), although occasionally, cells were found (plated at 1 million cells/well) to have extended processes (FIG. 1A (right, top image)). Human brain tissue cells were seeded onto three-dimensional silk scaffolds, and the live/dead cell staining of three-dimensional silk scaffold-based cultures at 24 hour (hr) post-seeding indicated that a majority of cells survived the tissue dissociation process (FIG. 1A (right, center image)). Cells in N or N+E media were more neuron-like with spindly processes (FIG. 7A (first and third columns)). In comparison, FBS addition (“N+FBS”) promoted fibroblast-like cells in two-dimensional cultures (FIG. 7A (second column, bottom)). There were ˜10 live cell/cm² in N media versus ˜550 live cell/cm² in N+E media at division 21 (DIV 21) (n>10 assessed). Given the drastic decrease from the plating density of >100,000 cells/cm² (see Methods), quantitative analysis of live/dead stained wells was not performed.

### Example 4

### Two-Dimensional Cultures of Human Patient Brain Tissue

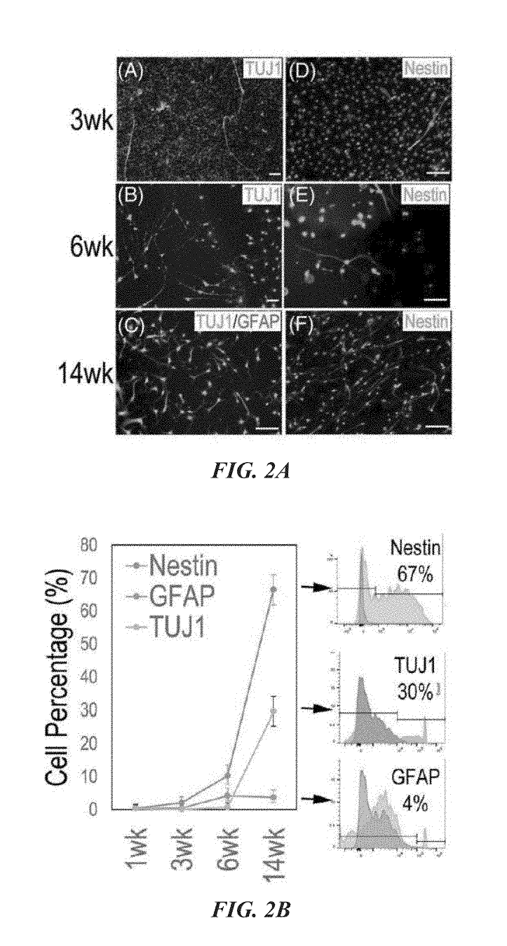

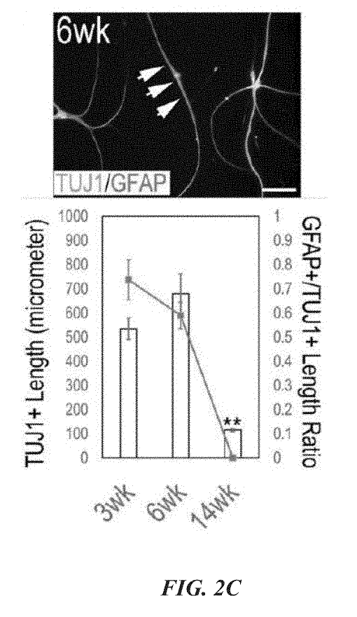

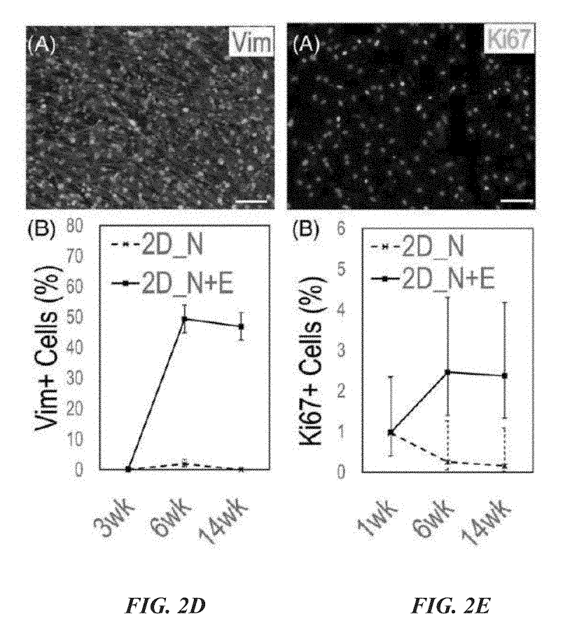

To determine cell phenotype changes in two-dimensional cultures, Cell marker expression was examined by immunofluorescence staining and flow cytometry over DIV 14 wks, including neuron-specific class III beta-tubulin (TUJ1-neuron), glial fibrillary acidic protein (GFAP-astrocyte), Nestin (neural stem/progenitor cell), and Vimentin (Vim) (neuroepithelial cell), (FIGS. 2A-2E). GFAP is expressed in mature astroglial cells. TUJ1 is expressed by both immature and mature neurons. Because few cells were present in two-dimensional cultures in the N media, all the images in FIGS. 2A-2E were from N+E cultures.

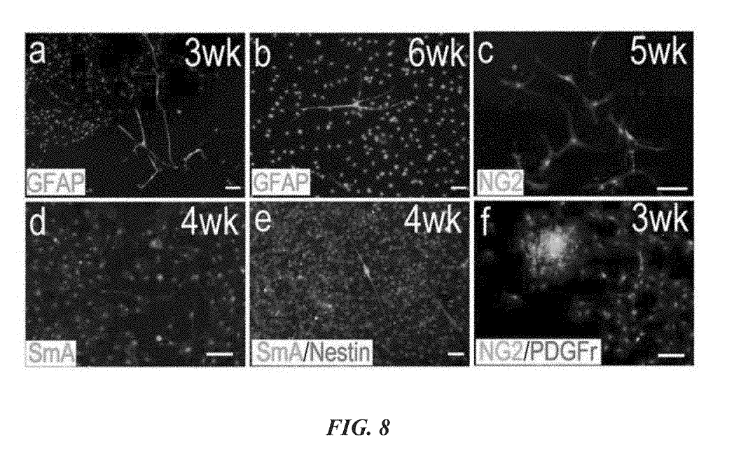

FIG. 2A shows TUJ1+, GFAP+, and Nestin+ cell morphology. FIG. 2B shows cell percentage changes of two-dimensional cultures in N+E media. At DIV 3 weeks, few cells were TUJ1+ (FIG. 2A (top, left)) or GFAP+ (FIG. 8 (top, left and center images)), less than 1% for either cell type as measured by flow cytometry (FIG. 2B). GFAP+ cells remained at a low level of ˜4% at DIV 6 and 14 weeks, with no significant differences between the time points (i.e., with overlapping 95% confidence intervals, FIG. 2B (left)). TUJ1+ cells increased from <1% at DIV 6 weeks to ˜30% at DIV 14 weeks (FIG. 2A (center left, bottom left), morphology; FIG. 2B, percentage). Nestin+ cells showed increasing percentages of ˜2% at DIV 3 weeks to ˜10% at DIV 6 weeks and ˜67% at DIV 14 weeks (FIG. 2A (right), morphology; FIG. 2B, percentage). The cell percentages at the two later time-points of TUJ1+ and Nestin+ cells were significantly different based on the non-overlapping 95% confidence intervals (FIG. 2B, DIV 6 weeks vs. DIV 3 weeks, DIV 14 weeks vs. DIV 6 weeks).

TUJ1+ cells at earlier time points (≤DIV 6 weeks) showed morphological differences compared to TUJ1+ cells at later time points (FIG. 2A (top left) vs. FIG. 2A (bottom left)). Additionally, TUJ1+ cells of early time points had longer lengths than later time points, of 533±45 μm (n=9), 679±81 μm (n=15), and 114±6 μm (n=24) at DIV 3, 6, and 14 weeks, respectively, with significant differences of DIV 14-weeks compared with DIV 3 and 6 weeks (analysis of variance test, p<0.001; FIG. 2C (bottom)).

Interestingly, some TUJ1+ processes were also positive for GFAP, with the co-staining pattern at different segments along the length (FIG. 2C (top), arrows). These TUJ1+/GFAP+ cells were bi-polar with spindly processes of relatively long lengths, (524±55 μm (n=20)), and a morphology similar to the neuroglial or radial glial cells associated with the embryonic brain (Draberova et al., 2008). In contrast, TUJ1+ cells at >DIV 6 weeks were negative for GFAP and mature neuron marker (MAP2), with short processes, indicating immature neuron phenotype (FIG. 2A (bottom left)).

Surprisingly, in cultures of later time points (≥DIV 6 weeks), Vim+ cells were found (FIG. 2D). Flow cytometry measured a burst increase of Vim+ cells from <0.5% at DIV 3 weeks to ˜50% at DIV 6 weeks, with significant difference of cell percentages at DIV 6 vs. 3 weeks and no significant difference at DIV 14 vs. 6 weeks (FIG. 2D (top), morphology; FIG. 2D (bottom), percentage). Other types of cells in N+E culture stained weakly positive for smooth muscle actin (SmA), but strongly positive for NG2 (neural/glial progenitor cell marker) and negative for CD31 (endothelial cell marker), PDGF receptor beta (pericyte marker; FIG. 8 (top right and bottom row)). Cell mitotic marker Ki67 showed negligible levels in cultures in N media and modest activity of ˜2% in N+E media at DIV 6 and 14 weeks, with no significant differences (FIG. 2E (top), morphology; FIG. 2E (bottom), percentage).

These data suggested that in two-dimensional cultures in N+E media, there was delayed expansion of Vim+ cells, Nestin+ neural stem/progenitor cells, and TUJ1+ immature neuron production that occurred at ≥DIV 6 weeks.

### Example 5

### Three-Dimensional Silk Scaffold-Based Culture Viability and DNA Content

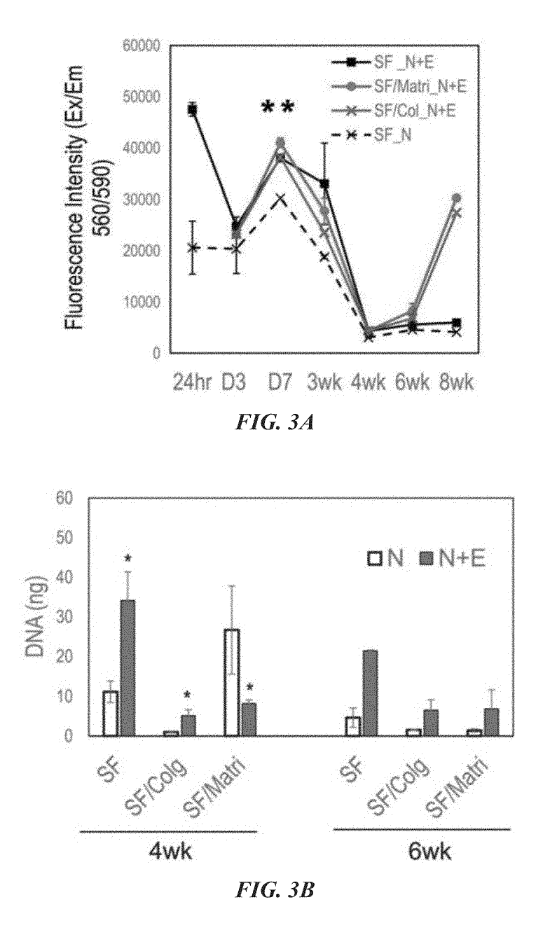

AlamarBlue® assay was used to evaluate three-dimensional culture viability (FIG. 3A). three-dimensional silk scaffold-based cultures (SF) in N+E media had higher tissue viability than in N media (SF_N+E vs. SF_N), with significant difference at DIV 7. In three-dimensional cultures of silk scaffold infused with collagen (SF/Col) or Matrigel mixed with collagen at 1:1 (SF/Matri), the ECM addition did not show significant improvement of tissue viability compared with SF cultures until DIV 6 weeks. After DIV 6 weeks, tissue viability started to increase, to >200% in the ECM-infused three-dimensional cultures.

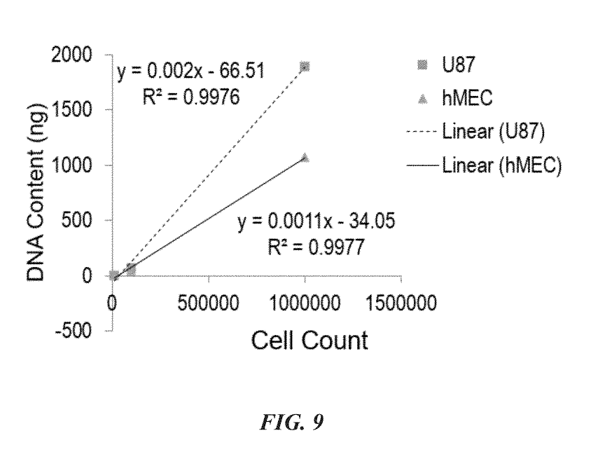

DNA content of extracted three-dimensional cultures was used to calculate total cell numbers (FIG. 3B). Three-dimensional cultures in N+E media had significantly higher DNA content than in N media (FIG. 3B, *, p<0.05, n=3-4/group). Cell numbers were estimated by extrapolation based on the linear curve of DNA-cell numbers generated with human brain-derived cell lines, such as the human microvascular endothelial cells hMEC/D3 and glioblastoma cells U87 (FIG. 9). It is estimated that there were ˜30,000-57,000 cells per three-dimensional culture in N+E media at DIV 4-6 weeks.

### Example 6

### Human Patient Brain Cells in Three-Dimensional Silk Scaffold-Only Cultures

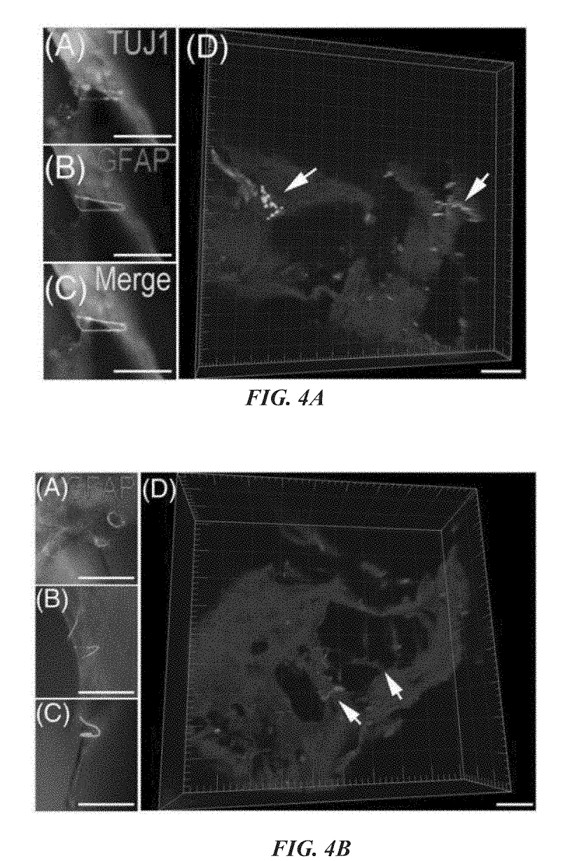

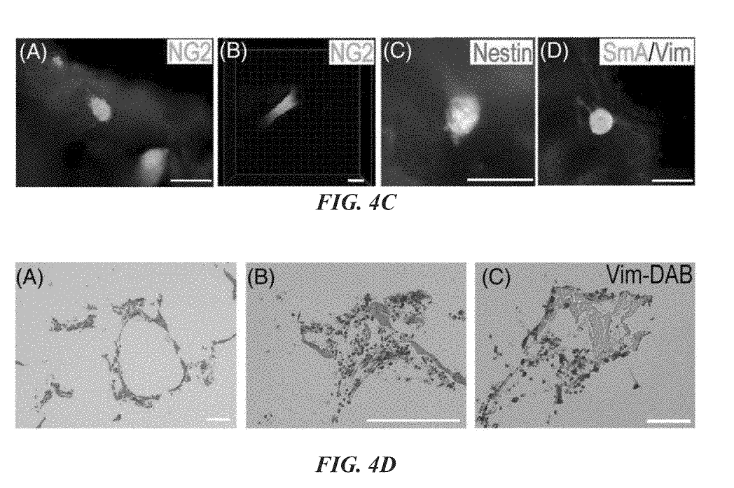

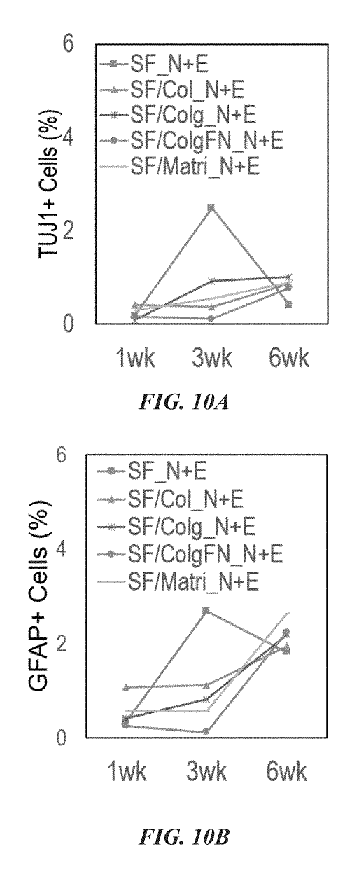

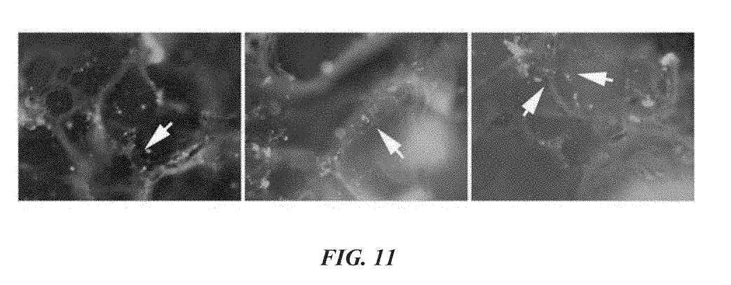

TUJ1+ staining in three-dimensional silk scaffold-based cultures showed fragmented patterns as early as DIV 1 week, indicating that most resident neurons were dead shortly after seeding (FIG. 4A). Unlike embryonic astrocytes that extended processes along the silk scaffold structure (Tang-Schomer et al., 2014), the mature human brain tissue-derived GFAP+ processes displayed a tendency to wrap around rod-shaped (diameter 5-30 μm) scaffold structures (FIGS. 4A, 4B, white arrows). A similar GFAP+ wrapping pattern was also found in three-dimensional silk scaffold-based cultures from the first brain tissue sample (FIG. 11). Both TUJ1+ and GFAP+ cells had low cell percentages (<3%) in all three-dimensional cultures as measured by flow cytometry (FIGS. 10A and 10B). Spheroid-looking cell clusters in three-dimensional cultures were positive for the neural progenitor cell markers NG2, nestin, and Vimentin (vim) at DIV 4-6 week's(FIG. 4C). Histological examination of paraffin sections of DIV 4-6 week's three-dimensional cultures showed cell clusters attached to the scaffold surface, and most cells were positive for Vim (FIG. 4D (left and center) H&E; FIG. 4D (right), Vim-DAB).

### Example 7

### Human Patient Brain Cells in Three-Dimensional Cultures of Silk Scaffolds Infused with ECM Gels

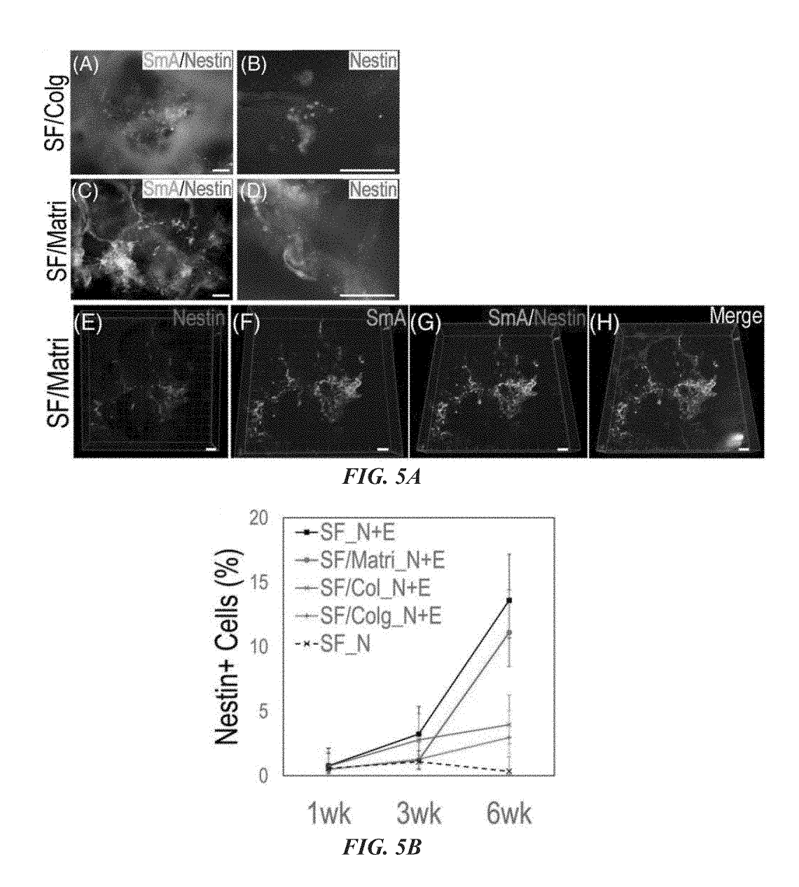

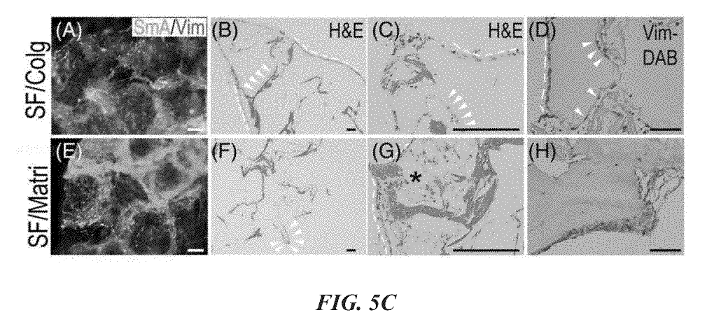

In three-dimensional cultures of silk scaffolds infused with ECM gels, Nestin+ cells showed cellular extensions into the matrix of SF/Matri cultures (FIG. 5A (center and bottom rows)), compared with a lack of cell outgrowth in collagen-infused three-dimensional cultures (FIG. 5A (top row)).

SF-only in N+E (SF_N+E) and SF/Matri in N+E (SF/Matri_N+E) cultures showed increasing Nestin+ cell numbers over time (FIG. 5B). For both SF_N+E and SF/Matri_N+E, the percentages at DIV 3 and 6 weeks were significantly different between the time points based on the non-overlapping 95% confidence intervals. Nestin+ cells in collagen-based three-dimensional cultures (SF/Col_N+E, SF/Colg_N+E) remained at a low percentage (<4%), with no significant changes over time. All three-dimensional cultures in N media had negligible Nestin+ cell percentages over time (<1%).Vim+ cells populated SF/Colg and SF/Matri three-dimensional cultures (FIG. 5C (first column)). A congruous cell sheet was found surrounding the outer surface of the scaffold (FIG. 5C, dotted line), as well as forming the interface (arrowhead) between the scaffold and the matrix (H&E, FIG. 5C (second and third columns)). The sheet-like structure was composed of cells positive for Vim (Vim-DAB, FIG. 5C (fourth column)).

These data demonstrated similar cell phenotype changes in three-dimensional silk scaffold-based cultures as in two-dimensional cultures, including improved cell viability in N+E versus N media, loss of endogenous neurons and glial cells and delayed expansion of neuroepithelial-like and neural stem/progenitor cells. In addition, the three-dimensional silk scaffold-based structure enabled more in vivo-like cell morphology, including the spiral wrapping of endogenous glial cells, and Vim+ neuroepithelial-like sheet formation at tissue boundaries.

### Example 8

### Human Patient Brain Cells in Suspension Cultures as Tissue Spheroids

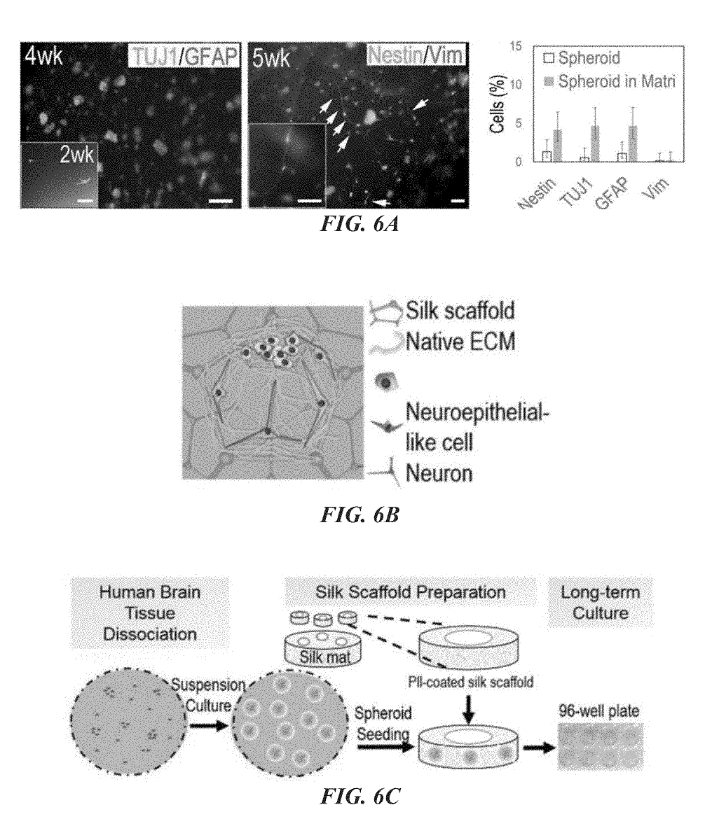

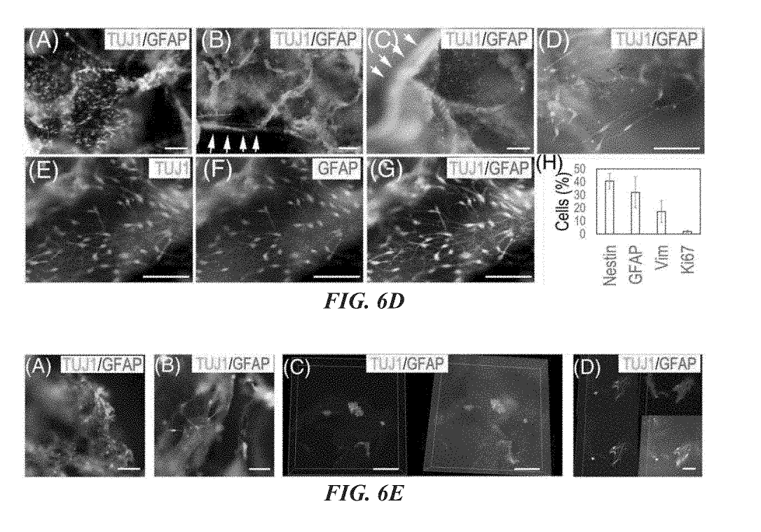

Tissue spheroids embedded in different types of ECM components were examined to determine whether a synthetic scaffold such as silk material is necessary for three-dimensional tissue formation (FIG. 6A). TUJ1+ and GFAP+ cells were found in spheroids embedded in Matrigel (Matri) in N or N+E media, but not in collagen-based matrix in either media (n>20 examined). TUJ1+ and GFAP+ cells remained as clusters, and no extended processes were found in the Matri group (FIG. 6A (left)). Short GFAP+ processes (<50 μm) were found in spheroid cultures in collagen-based matrix or Matri, in N or N+E media, at early time-points (<DIV 3 weeks; FIG. 6A (left), inset), but not at later time-points (n>20 examined), suggesting the early presence of residual astrocyte processes and their subsequent loss.

Nestin+ or Vim+ cells were found in spheroids embedded in Matri and maintained in N+E media (FIG. 6A (center)), not in N media, nor any spheroids embedded in collagen-based matrix (Col, Colg, ColgFN) in N or N+E media (n>20 examined). These cells had a bipolar shape with extended processes (FIG. 6A (center), arrows and inset). No discernable larger-scale structural formation was found in any of the spheroid cultures (n>50 examined).

Flow cytometry analysis of DIV 3 weeks cultures showed that in N+E media, spheroids embedded in Matri had more TUJ1+, GFAP+, and Nestin+ cells (˜4% for all three types), compared with spheroid-only cultures (FIG. 6A (right)); the differences were significant based on their non-overlapping 95% confidence intervals. However, both types of spheroid cultures had similarly negligible levels of Vim+ cells (<0.5%).

These data suggest that Matrigel in N+E media supported neural progenitor cell (Nestin+) growth but was insufficient for neuronal or glial outgrowth from human patient brain tissue spheroids. Compared with two-dimensional and three-dimensional silk scaffold-based cultures, these data further indicate that the presence of a stiff substrate was beneficial for inducing Vim+ neuroepithelial-like cell growth and cell-sheet formation.

Together, the above findings showed that the heterogeneous cell populations contained in the human patient brain tissue were capable of cell phenotype changes as direct responses to microenvironmental cues, such as medium, substrate form (flat vs. three-dimensional), and the surrounding matrix.

### Example 9

### Regeneration-Like Neuronal Growth of Human Patient Brain Tissue in Three-Dimensional In Vitro Culture

In development, neuroepithelial cells differentiate into radial glial cells that form the architectural paths for cells to migrate and further differentiate into neurons and astrocytes (Gotz & Huttner, 2005). The shared marker expression (Vim, Nestin, and NG2) of the cells derived from human patient brain tissue with neural/glial progenitor cells in development suggested remnant features to their embryonic precursors. We hypothesized that tissue spheroids derived from human patient brain tissue could maintain the activities of native tissue components in improved media conditions such as N+E and could respond to three-dimensional spatial cues such as provided by a three-dimensional silk scaffold and initiate regeneration-like tissue growth. FIG. 6B is the hypothetical illustration of different cell types of a neuronal lineage arising from a tissue spheroid that is housed within a three-dimensional scaffold. Tissue spheroids that had been maintained for 6 weeks in suspension culture were harvested, plated them into polylysine-coated three-dimensional silk scaffolds, and cultured for another 6-8 weeks in N+E media (FIG. 6C).

A large number of neuron-like cells in three-dimensional cultures were found (FIG. 6D). TUJ1+/GFAP+ cells covered regions of the scaffold surface (FIG. 6D (top, first three images)) and spread across pores (FIG. 6D (top, fourth image)). Cell sheet-like folds were observed near the periphery of the scaffolds (FIG. 6D (top, second and third images), arrows). Flow cytometry measured cell percentages of Nestin+, GFAP+, Vim+, and Ki67+ to be 41±6%, 32±12%, 17±8%, 2±1% (N=3; FIG. 6D (bottom, fourth image)).

To further differentiate neurons, another group of 6-week-old tissue spheroids in suspension culture were seeded into silk scaffolds and cultured in N+E for 2 weeks, then switched to the neural differentiation media NDM+GFs for another 6 weeks (FIG. 6E). TUJ1+ and GFAP+ cells were found in clusters (FIG. 6E (third image)) with extended processes (FIG. 6E (fourth image)).

In vitro cultures in defined chemical environment allow optimization of soluble factors. Serum-containing cultures invariably induced fibroblast-like cell growth that eventually overwhelmed the culture, with a similar cell morphology to the early reports of culture of adult human brain tissue in serum-containing medium (Gilden et al., 1975, 1976; Wroblewska et al., 1975). The chemically defined media, N+E (Neurobasal/B27/FGF/EGF mixed with VEGF-containing endothelial growth medium EGM-2), was identified to support long-term viability of human patient brain tissue-derived cultures. The Neuralbasal medium was originally developed for osmolality and the B27 supplement containing essential fatty acids, antioxidants, vitamins, and hormones (Brewer et al., 1993). The N media in this study also included FGF and EGF to support potential neural stem cells in the brain tissue (Guo, Patzlaff, Jobe, & Zhao, 2012). A classic tropic factor for cortical neurons (Morrison, Sharma, Vellis, & Bradshaw, 1986), FGF-2 is known to improve viability of isolated adult neurons in culture (Brewer, 1997; Brewer et al., 2001). However, the FGF-containing N media failed to support tissue growth in any of the in vitro systems examined in the study.

One key finding is that inclusion of VEGF-containing pro-endothelial cell condition (EGM-2 medium) enhanced cell viability in both two-dimensional and three-dimensional silk scaffold-based cultures of human patient brain tissue, and in particular, growth of neuroepithelial-like and neural progenitor cells. The pro-neural growth effect of EGM-2 is unlikely from resident endothelial cells, as CD31+ endothelial cells were absent in all cultures examined. As the only factor in EGM-MV that is absent in NeuralBasal/B27, VEGF is likely the contributing trophic factor to the regeneration-like in vitro growth. This finding is consistent with the pro-neurogenesis effect of VEGF in rodent studies, regarding its role in supporting neurogenesis and neuroepithelium development (Darland et al., 2011; Jin et al., 2002). It is unclear at present the effects of other components in the combined N+E media with respect to different types of cell responses of the human brain tissue. A more detailed study is needed to further refine medium supplements and incorporate additional microenvironmental factors such as sub-atmospheric oxygen levels for enhanced survival of primary human neurons (Kaplan et al., 1986).

The spheroid form is critical to preserve brain cell viability and cell phenotype responses to the three-dimensional environment. Viable cells in three-dimensional cultures were found to be contained only in spheroid-looking cell clusters. Spheroids previously maintained for 6 weeks in suspension culture were able to produce substantial cell outgrowth and expansion into a cell sheet-like structure after seeding into three-dimensional silk scaffolds. The spheroid form is known to preserve the pluripotency of many stem/progenitor cell types, including human embryonic stem cells, iPSCs, mesenchymal stem cells, and progenitor cells of various tissue types (Milet & Monsoro-Burq, 2012; Takai et al., 2016; Yamaguchi, Ohno, Sato, Kido, & Fukushima, 2014). It is important to note that the micro tissue-comprised spheroids used are different than the spheroids consisting of single cell aggregates in stem cell studies. It is unlikely that hypoxia and necrosis associated with rapid stem/progenitor cell expansion commonly described for three-dimensional spheroid cultures would occur in spheroids, as these micro brain tissue (<100 μm) did not grow in size in suspension culture and grew slowly in the three-dimensional systems. Aggregates of reassembled single cells from dissociated human brain tissue failed to survive in culture.

These findings suggest that the role of the micro-tissue spheroid form may be the preservation of native cell-cell and cell-ECM interactions of the human patient brain tissue, as well as endogenous responses to in vivo-like microenvironmental cues.

The presence of the three-dimensional scaffold was found to be important to support in vitro tissue-like structural formation from human patient brain cells. Vim+ cells formed a cell sheet in three-dimensional silk scaffold-based cultures, but not in scaffold-free Matrigel-based spheroid cultures. Moreover, the resident astroglial cells wrapped around rod-shaped three-dimensional scaffold structures that is similar to the in vivo astrocyte interaction with microvessels (Bonomini & Rezzani, 2010); such a morphology was not observed in two-dimensional cultures or ECM gel-based cultures. It is unclear why extracellular matrix gel-based systems failed in supporting Vim+ cell expansion and cell sheet formation. One potential mechanism may involve degradation of hydrogels and subsequent structural collapse that can negatively impact brain cell regenerative growth. Nevertheless, these features indicate that the cells in isolated human patient brain tissue can respond to three-dimensional structural cues and form their preferred arrangements akin to their in vivo correlates. The Vim+ neuroepithelium-like sheet was found to form at tissue boundaries of the three-dimensional cultures. However, in most studies of iPSC-derived three-dimensional cultures, the developing neuroepithelium was located near the center of a brain organoid (Lancaster et al., 2013; Lee et al., 2017). The inside-out fashion of neuroepithelium formation and associated axon radial growth in these brain organoids is reminiscent of the developmental neurogenesis program independent of a supporting scaffold. In contrast, the three-dimensional scaffold-based human brain tissue growth in this study responded to biophysical factors such as the presence of a scaffold and its microstructures. These differences suggest distinction between in vitro growth of reprogrammed progenitor cells versus regenerative process of primary human brain cells.

A surprising finding of is that the in vitro growth of human patient brain tissue showed regeneration-like progression of cell phenotype changes. There was delayed expansion of neuroepithelial-like and neural progenitor cells in substrate-supported cultures in two-dimensional and three-dimensional, at around DIV 6 weeks. Newly generated neurons arose at a further delayed time-point, that is, DIV 14 weeks, in both two-dimensional and three-dimensional cultures. The human patient brain tissue-derived cultures showed features that are typically only associated with the embryonic brain, such as TUJ1+/GFAP+ double-stained cells (Draberova et al., 2008) and neuroepithelium-like structure, suggesting responses of neural precursors to the in vitro environment. The human neocortex tissue used in the study is thought to lack adult neurogenesis (Rakic, 2002). The brain tissue samples used in the study were obtained from the neocortex area superficial to the epileptogenic temporal lobe region, unlikely to be contaminated by neurogenic areas of the brain, such as the subventricular zone, the subependymal zone, and the hippocampus (Kirby, Kuwahara, Messer, & Wyss-Coray, 2015). Though progenitor cells have been reported to exist in numerous brain areas and suggested to be capable of transitioning from quiescent state to responding to brain injuries (Bonfanti, 2013). There are conflicting results of neocortex neurogenesis studies, in part due to challenges of in vivo cell lineage studies with regard to spatial resolution and cell marker specificity. It is possible that the in vitro three-dimensional regeneration-like growth may derive from quiescent adult neural stem cells that were contained in the dissected neocortex tissue and had too few numbers to be detected. This could explain the initial low levels and subsequent delayed increases of Nestin+ cells in culture. However, many questions remain with regard to the potential cell of origin of the in vitro regeneration-like growth, the developmental state of the newly produced neurons and GFAP+ cells, and the role of the neuroepithelial-like cell sheet.

The three-dimensional model's ability to support viable neuronal cell expansion from a patient's normal brain tissue is a step forward from lack of primary human brain cell culture in the past. More detailed characterization of the three-dimensional culture system is needed for further model development. For example, electrophysiological evaluation such as whole cell recording would be necessary for evaluating the functional state of the newly generated neuron. Neuronal type specific markers, for example, GABAergic or glutamatergic, should be included in future cell type analysis of three-dimensional neuronal cultures. In addition, lack of MAP+ mature neurons suggests that the three-dimensional brain tissue model needs further optimization. Recently, Exogenous electrical signals have been demonstrated to promote three-dimensional axon growth and alignment of rat embryonic cortical neurons (Tang-Schomer, 2018). These novel approaches could provide temporally controlled biophysical signals to support brain tissue maturation in vitro. Moreover, the importance of VEGF-containing pro-endothelial growth condition points to the essential role of the vascular system in the brain. Incorporating perfusable blood vessel and microvascular network into the three-dimensional brain tissue model will allow for neuro-vascular interactions and development of the blood-brain barrier. This feature is particularly important for drug screening and brain disease modelling. Technologies for integrating these elements need to be developed in order to realize the potential of in vitro three-dimensional brain tissue models for recapitulating complex human brain functions and disorders.

The main objectives in epilepsy surgery are removal of the epileptogenic tissue and avoidance of surgical morbidity. In many cases, removal of a small rim of normal-looking cortex tissue surrounding the epileptic legion is warranted for seizure control. This tissue is often discarded under current clinical practice. The three-dimensional culture system with cells from epilepsy surgical patient's brain tissue provides an excellent opportunity for developing “normal” brain tissue model. Expanding the patient pool to include those from other centers could greatly facilitate further model development work. Once fully optimized and validated, the three-dimensional brain model with epilepsy surgical patient's tissue could provide a novel tool to study cellular mechanisms of epilepsy. If extended by incorporating electrical stimulation and recording, this personalized model could provide an exciting opportunity to, someday, compare a patient's neural network activity in vitro to the patient's clinical electrophysiological findings and to test drugs for treatment options.

### Methods

Human Patient Brain Tissue

Human patient brain tissue was obtained from epilepsy neurosurgery in CCMC at Hartford, Conn., USA. The procedures were approved by the Institutional Research Boards of UConn Health Center and CCMC (IRB #15-033). Informed consent was obtained from all human patients prior to the surgery. All methods were performed in accordance with the guidelines and regulations by the approved IRB protocol. Tissue specimen was transported in chilled RPMI-1640 medium (Sigma-Aldrich, St Louis, Mo., USA) containing 1% penicillin-streptomycin (Pen/Strep, Thermo Fisher, Waltham, Mass., USA) on ice pack from the operation room to the laboratory in <4-hr postsurgery.

Brain Tissue Dissociation

The tissue specimens were weighed, cut into ˜1 mm³ pieces with a sterile razor blade, resuspended at 1,600 mg tissue/10 ml in Hibernate-A medium (Thermo Fisher) containing 1% Pen/Strep and primocin (10 μg/ml, InvivoGen, San Diego, Calif., USA). The tissue suspension was transferred to gentleMACS C tubes (Miltenyi Biotec, San Diego, Calif., USA) and mounted onto gentleMACS Octo Dissociator (Miltenyi Biotec). Three cycles of centrifugation with machine-installed “human-tumour” protocols were used. Cell dissociates were filtered with 100-μm cell strainer (Fisher Scientific, Suwannee, Ga., USA). Approximate 30,000-100,000 cells/mg tissue were obtained.

Silk Protein-Based Scaffolds and ECM Gel Preparation

Silk solution and porous scaffolds were prepared from B. mori cocoons as described previously (Tang-Schomer et al., 2014). Salt-leached porous silk mats of 100 mm diameter were provided by David Kaplan's laboratory at Tufts University. A biopsy punch was used to generate donut-shaped scaffolds (outer diameter, 5 mm; inner diameter, 2 mm; height, 2 mm). Silk scaffolds were autoclaved, coated with poly-L-lysine (10 μg/ml, Sigma) overnight, and washed 3 times with phosphate buffered saline (PBS, Sigma).

Collagen gel was prepared from high-concentration rat tail type I collagen (8-10 mg/ml, Fisher Scientific), 10X M199 medium (Thermo Fisher) and 1 M sodium hydroxide mixed at a ratio of 88:10:2, followed by gelling at 37° C. for 1-2 hr. Other types of collagen gel-based matrix include collagen type I supplemented with EGM-2 MV SingleQuot (Lonza) without FBS (including hydrocortisone, hFGF-B, 2 ng/ml VEGF, R3-IGF-1, ascorbic acid, and hEGF) (“Colg”), and Colg supplemented with 10 μg/ml fibronectin (Fisher Scientific) (“ColgFN”). Matrigel (˜10 mg/ml, growth factor reduced, Fisher Scientific) was mixed at 1:1 ratio with collagen gel before infusing the silk scaffolds.

To make a scaffold-gel composite structure, the scaffolds were washed with ECM gels in the liquid form to replace the medium within the scaffold, followed by incubation at 37° C. for 1-2 hr before culture medium immersion. To make tissue spheroid-gel structure, approximately 50-μl matrix gel solution was used to embed the spheroids in U-shaped wells of a 96-well plate.

### Cell Plating

For two-dimensional cultures, cells were plated at 105,263 cells/cm² in six-well plate (corresponding to 1 million cells/well). One plate per time-point was used, totaling 14 plates. For three-dimensional scaffold-based cultures, the scaffolds were immersed in high-density cell suspensions (˜100 million cells/ml) for 24 hr followed by extensive washes with media and proceed to scaffold-only cultures or ECM gel-infused composite cultures. For scaffold-free cultures, cell dissociates were distributed at ˜40,000 cells/well to U-shaped wells of a 96-well plate. At 3 days in vitro (DIV 3), some three-dimensional cultures and spheroids in suspension cultures were embedded in ECM matrix.

Culture media used include NeuralBasal/B27 (Invitrogen, Grand Island, N.Y., USA) supplemented with 20 ng/ml recombinant human fibroblast growth factor, basic-154 (FGF, ConnStem, Cheshire, Conn., USA) and 20 ng/ml human epidermal growth factor (EGF, PeproTech, Rocky Hill, N.J., USA), termed “N” medium; NeuralBasal/B27/EGF/FGF supplemented with 10% fetal bovine serum (FBS, Denville Scientific, Metuchen, N.J., USA) (“N+FBS”), and “N” medium mixed at 1:1 with endothelial growth media EGM-2MV (Lonza, Walkersville, Md., USA) without serum (“N+E”); Neuralbasal/B27 supplemented with 10 ng/ml recombinant human glial-derived neurotrophic factor (GDNF) (PeproTech) and 10 ng/ml recombinant human brain-derived neurotrophic factor (BDNF) (R&D Systems, Minneapolis, Minn., USA)(“N+GFs”); Neuralbasal/B27 supplemented with cyclic adenosine monophosphate (cAMP, 100 ng/ml, Sigma), ascorbic acid (1 μg/ml, Sigma), GDNF (10 ng/ml), and BDNF (10 ng/ml) (“NDM+GFs”). Media were changed once a week for all culture systems.

Tissue Viability Assay

AlamarBlue® assay was used to measure cell viability of three-dimensional cultures, according to the manufacturer's protocol (ThermoFisher Scientific). Briefly, alamarBlue® reagent was mixed in fresh culture media (1:10, v/v) and added to the cells in three-dimensional cultured and incubated for 2 hr at 37° C. The solution was transferred into a new 96-well plate. The fluorescence intensity was read at Ex/Em of 560/590 nm on a micro-plate spectrophotometer (SynergyMx Gen5, BioTek, Winooski, Vt., USA). Three to four replicate cultures per group per time-point were used for this assay.

Live/Dead Assay

Live/dead assay was used to image live cells in cultures, according to the manufacturer's protocol (ThermoFisher Scientific). Briefly, cultures were washed once with PBS, incubated with fresh medium containing calcein-AM (Live stain, 2 μg/ml) and ethidium homodimer-1 (Dead stain, 1 μg/ml) at 37° C. for 20 min, washed with PBS once, and returned to fresh medium for another 20 min at 37° C. The stained cultures were imaged with a fluorescence microscope at Ex/Em of 494/517 and 528/617 nm for live and dead stains, respectively.

Flow Cytometry Cell Counting

Cells were treated with 0.05% trypsin-EDTA (5 min) (Invitrogen) and 0.25% trypsin-EDTA (10 min) for two-dimensional and three-dimensional cultures, respectively. Cell suspensions were mixed at 1:1 with medium containing 10%FBS and centrifuged at 300 g, 5 min. Cell pellets were resuspended in PBS containing 2% FBS and stained on ice for 15 min with eFluor 780 (Affymetrix eBioscience, San Diego, Calif., USA). Cells were washed in 2% FBS-containing PBS by centrifuging at 300 g, 5 min. Cell pellets were resuspended and stained with membrane-bound flow antibodies on ice for 20 min and washed. Stained/washed cells were fixed with 4% paraformaldehyde (Electron Microscopy Sciences, Hatfield, Pa., USA) for 20 min, washed, and permeabilized with PBS containing 0.1% Tween and 2% FBS for 20 min. Cells were subsequently stained with intracellular flow antibodies for 30 min, washed, and proceeded to flow cytometry.

Flow antibodies used include anti-human CD31-PE-Cy7 (BD Biosciences, San Jose, Calif. USA), anti-Nestin-Alexa 647 (BD Biosciences), anti-GFAP-Alexa 488 (eBioscience), anti-TUJ1-PerCP (R&D Systems), anti-Vimentin-PE (BD Biosciences), and anti-Ki67-eFluor 450 (eBioscience).

Flow cytometry cell counting was performed on a BD LSR II instrument equipped with five lasers with BD FACS DIVA software (BD Biosciences). One thousand cells per sample were counted and analyzed with FlowJo software (FlowJo, Ashland, Oreg., USA). Unstained cells were used to set a gate for “live & single” cells. eFluor 780-stained cells were used to set the gate for “live” cells and “control” gates for each stain with a threshold of 0.5%. Positive cell population corresponding to a stain was calculated from the multiplexed cell population using the same gate as that used for the control unstained cell population.

Immunofluorescence Staining and Imaging

Cell cultures were fixed with 4% paraformaldehyde (Electron Microscopy Sciences) for 20 min, washed, and permeabilized with PBS constaining 0.1% Triton X-100 (Fisher Scientific) and 4% normal goat serum (Jackson ImmunoResearch Labs, West Grove, Pa., USA) for 20 min, followed with incubation of primary antibodies overnight at 4° C. After three 10-min PBS washes, cells were incubated with secondary antibodies for 1 hr at room temperature, followed by extensive washes. Antibodies included anti-βIII-tubulin (TUJ1, mouse clone 2G10, 1:500, eBioscience; rabbit, Sigma), anti-glial fibrillary acidic protein (GFAP, mouse clone GAS, 1:500, eBioscience; rabbit, Thermo Fisher), anti-Nestin (mouse clone 10C2, 1:100, eBioscience), anti-Ki67 (mouse clone B56, 1:100, BD Biosciences), anti-Vimentin (mouse clone RV202, 1:200, BD Bioscience), anti-alpha smooth muscle actin (SmA, rabbit clone E184, 1:100, Abcam, Cambridge, Mass. USA), anti-microtubule associated protein-2 (MAP2, mouse clone M13, 1:200, Life Technologies, Grand Island, N.Y., USA), anti-neural/glial antigen 2(NG2, mouse clone 9.2.27, 1:100, eBioscience), and anti-platelet-derived growth factor receptor beta (PDGF-RB, rabbit, 1:50, Thermo Fisher). Goat anti-mouse or rabbit Alexa 488 and 546 (1:250; Invitrogen) secondary antibodies were used. Fluorescence images were acquired on a Zeiss fluorescence microscope using excitation/emission (Ex/Em) of 493/520 nm for Alexa 488 and Ex/Em of 562/573 nm for Alexa 546. Confocal images were acquired on a Zeiss 780 laser scanning confocal imaging system.

Immunohistology

Three-dimensional cultures were fixed in 4% paraformaldehyde, washed, stored in 70% ethanol, and processed in Shandon Pathcentre (Thermo Fisher) and paraffin-embedded in HistoCentre 2 (GMI, Ramsey, Minn. USA). Five-micrometre sections were cut with a microtome (Olympus CUT 4055) and mounted onto poly-1-lysine (Sigma) coated glass slides. Some sections were proceeded to standard haematoxylin and eosin (H&E) staining. For antibody staining, sections were antigen-retrieved by immersing in a citric buffer (Thermo Fisher) inside a steamer at 95° C. for 20 min. Slides were incubated with mouse primary antibody solution at 4° C. overnight, followed by three 5-min PBS washes, blocked by hydrogen peroxide (3%, Thermo Fisher) for 10 min at room temperature, and washed 3× with PBS. Slides were stained with anti-mouse secondary antibodies with the ImmPress Reagent kit (Vector Laboratories, Burlingame, Calif. USA) for 1 hr at room temperature, washed, and treated with the DAB peroxidase substrate kit (Vector) for 3-5 min under a microscope. After extensive washes, slides were counter-stained with haematoxylin for ˜10 s, dehydrated, cleared, and mounted.

Picogreen DNA Quantification

Cell cultures were subjected to extraction with AllPrep DNA/RNA/Protein kit (Qiagen, Valencia, Calif., USA), as previously described (Tang-Schomer et al, 2014). DNA quantity was measured using a PicoGreen dsDNA Assay kit (Invitrogen). DNA-cell number correlation curves were derived from two-dimensional cultures of human cerebral microvascular endothelial cell line (hCMEC/D3, EMD Millipore, Billerica, Mass. USA).

Statistical Analysis

Data are mean ± standard error of mean, except where otherwise noted. Analysis used analysis of variance test. For all tests, p<0.05 was considered significant. For statistical analysis of flow cytometry-measured cell percentages, construction of simultaneous confidence intervals was performed (see Results). A programme wrote in R was used to implement the analysis.

### REFERENCES

### Each of Which is Incorporated Herein by Reference

Agresti, A., Bini, M., Bertaccini, B., & Ryu, E. (2008). Simultaneous confidence intervals for comparing binomial parameters. Biometrics, 64(4), 1270-1275. https://doi.org/10.1111/j.1541-0420.2008.00990.x
Agresti, A., & Coull, B. A. (1998). Approximate is better than “exact” for interval estimation of binomial proportions. The American Statistician, 52(2), 119-126. https://doi.org/10.2307/2685469
Altman, G. H., Diaz, F., Jakuba, C., Calabro, T., Horan, R. L., Chen, J., Kaplan, D. L. (2003). Silk-based biomaterials. Biomaterials, 24(3), 401-416.
Bonfanti, L. (2013). The (real) neurogenic/gliogenic potential of the postnatal and adult brain parenchyma. ISRN Neuroscience, 2013, 354136-354114. https://doi.org/10.1155/2013/354136
Bonomini, F., & Rezzani, R. (2010). Aquaporin and blood brain barrier. Current Neuropharmacology, 8(2), 92-96. https://doi.org/10.2174/157015910791233132
Brewer, G. J. (1997). Isolation and culture of adult rat hippocampal neurons. Journal of Neuroscience Methods, 71(2), 143-155. doi:S0165-0270(96)00136-7 [pii]
Brewer, G. J., Espinosa, J., Mcllhaney, M. P., Pencek, T. P., Kesslak, J. P., Cotman, C., McManus, D. C. (2001). Culture and regeneration of human neurons after brain surgery. Journal of Neuroscience Methods, 107(1-2), 15-23. doi:S0165027001003429 [pii]
Brewer, G. J., & Torricelli, J. R. (2007). Isolation and culture of adult neurons and neurospheres. Nature Protocols, 2(6), 1490-1498. doi: nprot.2007.207 [pii]
Brewer, G. J., Torricelli, J. R., Evege, E. K., & Price, P. J. (1993). Optimized survival of hippocampal neurons in B27-supplemented neurobasalm, a new serum-free medium combination. Journal of Neuroscience Methods, 35, 567-576.
Brown, L. D., Cal, T. T., & DasGupta, A. (2001). Interval estimation for a binomial proportion, 101-133. https://doi.org/10.1214/ss/1009213286
Cairns, D. M., Chwalek, K., Moore, Y. E., Kelley, M. R., Abbott, R. D., Moss, S., & Kaplan, D. L. (2016). Expandable and rapidly differentiating human induced neural stem cell lines for multiple tissue engineering applications. Stem Cell Reports, 7(3), 557-570. https://doi.org/10.1016/j. stemcr.2016.07.017
Chwalek, K., Sood, D., Cantley, W. L., White, J. D., Tang-Schomer, M., & Kaplan, D. L. (2015). Engineered three-dimensional silk-collagen-based model of polarized neural tissue. Journal of Visualized Experiments: JoVE, (105). https://doi.org/10.3791/52970
Chwalek, K., Tang-Schomer, M. D., Omenetto, F. G., & Kaplan, D. L. (2015). In vitro bioengineered model of cortical brain tissue. Nature Protocols, 10(9), 1362-1373. https://doi.org/10.1038/nprot.2015.091
Darland, D. C., Cain, J. T., Berosik, M. A., Saint-Geniez, M., Odens, P. W., Schaubhut, G. J., D'Amore, P. A. (2011). Vascular endothelial growth factor (VEGF) isoform regulation of early forebrain development. Developmental Biology, 358(1), 9-22. https://doi.org/10.1016/j. ydbio.2011.06.045
Draberova, E., Del Valle, L., Gordon, J., Markova, V., Smejkalova, B., Bertrand, L., Katsetos, C. D. (2008). Class III Î2-tubulin is constitutively coexpressed with glial fibrillary acidic protein and nestin in midgestational human fetal astrocytes: Implications for phenotypic identity. Journal of Neuropathology and Experimental Neurology, 67(4), 341-354.
Eide, L., & McMurray, C. T. (2005). Culture of adult mouse neurons. BioTechniques, 38(1), 99-104. doi:05381RR02 [pii]
Giard, D. J., Aaronson, S. A., Todaro, G. J., Arnstein, P., Kersey, J. H., Dosik, H., & Parks, W. P. (1973). In vitro cultivation of human tumors: Establishment of cell lines derived from a series of solid tumors. Journal of the National Cancer Institute, 51(5), 1417-1423.
Gilden, D. H., Devlin, M., Wroblewska, Z., Friedman, H., Rorke, L. B., Santoli, D., & Koprowski, H. (1975). Human brain in tissue culture. I. Acquisition, initial processing, and establishment of brain cell cultures. The Journal of Comparative Neurology, 161(3), 295-306. https://doi.org/10.1002/cne.901610302
Gilden, D. H., Wroblewska, Z., Eng, L. F., & Rorke, L. B. (1976). Human brain in tissue culture: Part. 5. Identification of glial cells by immunofluorescence. Journal of the Neurological Sciences, 29(2-4), 177-184. https://doi.org/10.1016/0022-510X(76)90169-6
Gotz, M., & Huttner, W. B. (2005). The cell biology of neurogenesis. Nature Reviews Molecular Cell Biology, 6(10), 777-788. doi:nrm1739 [pii]
Gumnit, R., Labiner, D., Fountain, N., & Herman, S. (2012). Data on specialized epilepsy centers: Report to the institute of medicine's committee on the public health dimensions of the epilepsies. In M. England, C. Liverman, A. Schultz, & L. Strawbridge (Eds.), Epilepsy across the sprectrum. Washington D.C.: The National Academies Press.
Guo, W., Patzlaff, N. E., Jobe, E. M., & Zhao, X. (2012). Isolation of multipotent neural stem or progenitor cells from both the dentate gyrus and subventricular zone of a single adult mouse. Nature Protocols, 7(11), 2005-2012. https://doi.org/10.1038/nprot.2012.123
Jin, K., Zhu, Y., Sun, Y., Mao, X. O., Xie, L., & Greenberg, D. A. (2002). Vascular endothelial growth factor (VEGF) stimulates neurogenesis in vitro and in vivo. Proceedings of the National Academy of Sciences of the United States of America, 99(18), 11946-11950. https://doi.org/10.1073/pnas.182296499
Kaplan, F. S., Brighton, C. T., Boytim, M. J., Selzer, M. E., Lee, V., Spindler, K., Black, J. (1986). Enhanced survival of rat neonatal cerebral cortical neurons at subatmospheric oxygen tensions in vitro. Brain Research, 384(1), 199-203. https://doi.org/10.1016/0006-8993(86)91240-0
Kirby, E. D., Kuwahara, A. A., Messer, R. L., & Wyss-Coray, T. (2015). Adult hippocampal neural stem and progenitor cells regulate the neurogenic niche by secreting VEGF. Proceedings of the National Academy of Sciences of the United States of America, 112(13), 4128-4133. https:/doi.org/10.1073/pnas.1422448112
Lancaster, M. A., Renner, M., Martin, C. A., Wenzel, D., Bicknell, L. S., Hurles, M. E., Knoblich, J. A. (2013). Cerebral organoids model human brain development and microcephaly. Nature, 501(7467), 373-379. https://doi.org/10.1038/nature12517
Lee, C. T., Chen, J., Kindberg, A. A., Bendriem, R. M., Spivak, C. E., Williams, M. P., Freed, W. J. (2017). CYP3A5 mediates effects of cocaine on human neocorticogenesis: Studies using an in vitro three-dimensional self-organized hPSC model with a single cortex-like unit. Neuropsychopharmacology: Official Publication of the American College of Neuropsychopharmacology, 42(3), 774-784. https://doi.org/10.1038/npp.2016.156
Mattson, M. P., & Rychlik, B. (1990). Cell culture of cryopreserved human fetal cerebral cortical and hippocampal neurons: Neuronal development and responses to trophic factors. Brain Research, 522(2), 204-214. https://doi.org/10.1016/0006-8993(90)91462-P
Milet, C., & Monsoro-Burq, A. H. (2012). Embryonic stem cell strategies to explore neural crest development in human embryos. Developmental Biology, 366(1), 96-99. https://doi.org/10.1016/j.ydbio.2012.01.016
Morrison, R. S., Sharma, A., de Vellis, J., & Bradshaw, R. A. (1986). Basic fibroblast growth factor supports the survival of cerebral cortical neurons in primary culture. Proceedings of the National Academy of Sciences of the United States of America, 83(19), 7537-7541.
Rakic, P. (2002). Neurogenesis in adult primate neocortex: An evaluation of the evidence. Nature Reviews. Neuroscience, 3(1), 65-71. https://doi. org/10.1038/nrn700
Rockwood, D. N., Preda, R. C., Yucel, T., Wang, X., Lovett, M. L., & Kaplan, D. L. (2011). Materials fabrication from Bombyx mori silk fibroin. Nature Protocols, 6(10), 1612-1631. https://doi.org/10.1038/nprot.2011.379
Sood, D., Chwalek, K., Stuntz, E., Pouli, D., Du, C., Tang-Schomer, M. D., Kaplan, D. (2016). Fetal brain extracellular matrix boosts neuronal network formation in three-dimensional bioengineered model of cortical brain tissue. American Chemical Society, 2, 131-140. https://doi.org/10.1021/acsbiomaterials.5b00446
Takai, A., Fako, V., Dang, H., Forgues, M., Yu, Z., Budhu, A., & Wang, X. W. (2016). Three-dimensional organotypic culture models of human hepatocellular carcinoma. Scientific Reports, 6, 21174. https://doi.org/10.1038/srep21174
Tang-Schomer, M. D. (2018). three-dimensional axon growth by exogenous electrical stimulus and soluble factors. Brain Research, 1678, 288-296. doi:S0006-8993(17)30490-0 [pii]
Tang-Schomer, M. D., White, J. D., Tien, L. W., Schmitt, L. I., Valentin, T. M., Graziano, D. J., Kaplan, D. L. (2014). Bioengineered functional brain- like cortical tissue. Proceedings of the National Academy of Sciences of the United States of America, 111(38), 13811-13816. https://doi.org/10.1073/pnas.1324214111
Wroblewska, Z., Devlin, M., Gilden, D. H., Santoli, D., Friedman, H., & Koprowski, H. (1975). Human brain in tissue culture. II. Studies of long-term cultures. The Journal of Comparative Neurology, 161(3), 307-316. https://doi.org/10.1002/cne.901610303
Yamaguchi, Y., Ohno, J., Sato, A., Kido, H., & Fukushima, T. (2014). Mesenchymal stem cell spheroids exhibit enhanced in-vitro and in-vivo osteoregenerative potential. BMC Biotechnology, 14. 105-014-0105-9. https://doi.org/10.1186/s12896-014-0105-9

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1. A method comprising culturing human brain tissue spheroids on a three-dimensional scaffold in culture media comprising vascular endothelial growth factor (VEGF), and producing a population of cells, wherein cells of the population are positive for nestin, glial fibrillary acidic protein (GFAP), and/or vimentin.

2. The method of claim 1, wherein the three-dimensional scaffold comprises silk fibroin, optionally prepared from Bombyx mori cocoons.

3. The method of claim 1, wherein the three-dimensional scaffold is coated in polylysine, and/or wherein the three-dimensional scaffold further comprises extracellular matrix (ECM) components.

4. (canceled)

5. The method of claim 1, wherein the three-dimensional scaffold has a length, width, and/or diameter of 2 mm to 10 mm, and/or a height of 2 mm to 10 mm.

6. The method of claim 1, wherein the culture media further comprises Neurobasal™ Medium or an equivalent medium, and/or wherein the culture media further comprises endothelial growth medium (EGM™), optionally EGM™-2 MV, or an equivalent medium.

7. (canceled)

8. The method of claim 1, wherein the culture media further comprises a B-27 supplement, fibroblast growth factor (FGF), and/or epidermal growth factor (EGF).

9. The method of claim 1, wherein the culture media further comprises Neurobasal™ Medium, EGM™-2 MV, B-27 supplement, FGF, and EGF.

10. The method of claim 1, wherein the concentration of VEGF is 1-5 ng/ml, optionally 2 ng/ml.

11. The method of claim 1, wherein the concentration of FGF is 10-30 nm/mL, optionally 20 ng/ml, and/or wherein the concentration of EGF is 10-30 nm/mL, optionally 20 ng/ml.

12. The method of claim 1, wherein the human brain tissue spheroids have a diameter of less than or equal to 100 μm.

13. The method of claim 1, wherein the human brain tissue spheroids are obtained from a human neocortex or temporal lobe.

14. The method of claim 1, wherein the human brain tissue spheroids are cultured for at least 2 weeks, at least 4 weeks, or at least 6 weeks.

15. The method of claim 1, wherein at least 30-50% of the cells of the population are positive for nestin, at least 20-40% of the cells of the population are positive for GFAP, and/or at least 10-25% of the cells of the population are positive for vimentin.

16. The method of claim 1, further comprising replacing the culture media with neural differentiation media, optionally comprising Neurobasal™ Medium, B-27 supplement, cyclic adenosine monophosphate (cAMP), ascorbic acid, glial cell line-derived neurotrophic factor (GDNF), and/or brain-derived neurotrophic factor (BDNF).

17. The method of claim 16, wherein the concentration of cAMP is 50-150 ng/ml, the concentration of ascorbic acid is 0.5-2 μg/ml, the concentration of GDNF is 5-20 ng/ml, and/or the concentration of BDNF is 5-20 ng/ml.

18. The method of claim 1 further comprising culturing the three-dimensional scaffold and the population of cells in the neural differentiation media, optionally for at least 4 weeks, and producing cell clusters comprising cells that are positive for neuron-specific class III beta-tubulin (TUJ1) and GFAP.

19. The method of claim 1 any one of claims 1-18 further comprising dissociating a human brain tissue sample to obtain the human brain tissue spheroids.

20. The method of claim 19 further comprising culturing the human brain tissue spheroids in a suspension culture for at least 4 weeks, prior to culturing the human brain tissue spheroids on the three-dimensional scaffold.

21. A composition comprising human brain tissue spheroids, a three-dimensional scaffold, culture media comprising vascular endothelial growth factor (VEGF), and a population of cells, wherein cells of the population are positive for nestin, glial fibrillary acidic protein (GFAP), neuron-specific class III beta-tubulin (TUJI), and/or vimentin.

22.-34. (canceled)

35. A method comprising culturing human brain tissue spheroids on a three-dimensional scaffold in culture media comprising vascular endothelial growth factor (VEGF) in the presence of a test agent, and assessing the effect of the test agent on human brain tissue spheroids.
